(19) United States
(12) Patent Application Publication
(10) Pub. No.: US 2013/0266488 A1
(43) Pub. Date: Oct. 10, 2013

(54) CASSETTE FOR RADIOACTIVE ISOTOPE HANDLING APPARATUS, RADIOACTIVE ISOTOPE HANDLING APPARATUS, AND RADIOACTIVE ISOTOPE HANDLING SYSTEM

(71) Applicant: SUMITOMO HEAVY INDUSTRIES, LTD., (US)

(72) Inventors: Jun KATO, Niihama-shi (JP); Tomoya IWATA, Niihama-shi (JP); Takashi ODA, Niihama-shi (JP)

(73) Assignee: SUMITOMO HEAVY INDUSTRIES, LTD., Tokyo (JP)

(21) Appl. No.: 13/800,225

(22) Filed: Mar. 13, 2013

(30) Foreign Application Priority Data
Mar. 13, 2012 (JP) 2012-056070
Feb. 6, 2013 (JP) 2013-021428

(51) Int. Cl. A61K 51/00

(52) U.S. Cl. CPC A61K 51/00
USPC 422/256; 422/310

(57) ABSTRACT

A cassette for a radioactive isotope handling apparatus includes a substrate including a plurality of holders capable of attaching piping; and piping attached to the substrate by some of the plurality of holders. The substrate may be provided with a plurality of through holes for opening and closing the piping. The plurality of holders have a plurality of first holders capable of attaching the piping along a first direction; and a plurality of second holders capable of attaching the piping along a second direction intersecting the first direction.

### INCORPORATION BY REFERENCE

Priority is claimed to Japanese Patent Application No. 2012-056070, filed Mar. 13, 2012 and Japanese Patent Application No. 2013-021428, filed Feb. 6, 2013, the entire content of which are incorporated herein by reference.

### BACKGROUND

1. Technical Field

The present invention relates to a cassette for a radioactive isotope handling apparatus, a radioactive isotope handling apparatus, and a radioactive isotope handling system.

2. Description of the Related Art

For example, radioactive isotope labeled compounds (RI compounds) used for positron emission tomography examination (PET examination) in hospitals or the like are synthesized by RI compound synthesizing apparatuses that make radioactive isotopes (RI) chemically react with a predetermined raw material reagent. Such a synthesizing apparatus is disclosed in the related art. This synthesizing apparatus includes a fixed module, and a disposable module in which a plurality of pieces of piping is fixed to a substrate. In this synthesizing apparatus, if one synthesis is completed, a disposable module is replaced with a new one to prepare for the next synthesis.

### SUMMARY

According to an embodiment of the present invention, there is provided a cassette for a radioactive isotope handling apparatus which includes a substrate including a plurality of holders capable of attaching piping; and piping attached to the substrate by some of the plurality of holders, wherein the substrate is provided with a plurality of through holes for opening and closing the piping, at least two holders are provided corresponding to each of the through holes, and wherein the plurality of holders include a plurality of first holders capable of attaching the piping along a first direction; and a plurality of second holders capable of attaching the piping along a second direction intersecting the first direction.

According to another embodiment of the present invention, there is provided a radioactive isotope handling apparatus which includes a fixing portion capable of detachably fixing the cassette for a radioactive isotope handling apparatus; and a plurality of pressing members that are provided at positions facing the plurality of through holes, respectively, and are capable of pressing the piping.

According to still another embodiment of the present invention, there is provided a radioactive isotope handling system which includes a fixing portion capable of detachably fixing the cassette for a radioactive isotope handling apparatus; a plurality of pressing members that are provided at positions that face the plurality of through holes, respectively, and are capable of pressing the piping; a solution adjustment unit that performs adjustment of a solution in which radioactive isotopes are dissolved; and a refinement section that refines the radioactive isotopes contained in the solution adjusted in the solution adjustment unit, wherein the refinement section may have an extraction section that extracts the radioactive isotopes from the solution; a replaceable three-way stopcock that is provided downstream of the extraction section; and a drive section that is provided separately from the three-way stopcock, and applies a driving force for switching of the three-way stopcock.

### BRIEF DESCRIPTION OF THE DRAWINGS

FIG. 1 is a perspective view showing the configuration of a radioactive drug synthesizing apparatus illustrated as a radioactive isotope handling apparatus related to one embodiment of the invention.

FIG. 2 is a front view showing a configuration example of a plate that the radioactive drug synthesizing apparatus of FIG. 1 has.

FIG. 3 is a view showing a first configuration example of a detachable module using the plate of FIG. 2.

FIG. 4 is a view showing a second configuration example of the detachable module using the plate of FIG. 2.

FIG. 5 is a view showing a third configuration example of the detachable module using the plate of FIG. 2.

FIG. 6 is a view showing a fourth configuration example of the detachable module using the plate of FIG. 2.

FIG. 7 is a view showing a fifth configuration example of the detachable module using the plate of FIG. 2.

FIG. 8 is a schematic configuration view showing a system configuration of a radioactive isotope refining system including a solution adjusting apparatus illustrated as a radioactive isotope handling apparatus related to another embodiment of the invention.

FIG. 9 is a plan view showing an example of the configuration of a cassette to be used in the solution adjusting apparatus of FIG. 8.

FIG. 10 is a front view showing an example of the configuration of the solution adjustment unit shown in FIG. 8.

FIG. 11 is a front view showing a state where a door portion of the solution adjustment unit shown in FIG. 10 is opened.

FIG. 12 is a front view showing an aspect in which the cassette is fixed to a fixing portion of the solution adjustment unit shown in FIG. 11.

FIG. 13 is a cross-sectional view taken along line XIII-XIII shown in FIG. 12, and is a cross-sectional view in a state where the door portion is closed.

FIG. 14 is a schematic configuration view showing an example of the radioactive isotope refining system in a case where ⁶⁴Cu is refined.

FIG. 15 is a schematic configuration view showing an example of the radioactive isotope refining system in a case where ⁸⁹Zr is refined.

FIG. 16 is a schematic configuration view showing an example of the radioactive isotope refining system in a case where ^{99m}Tc is refined.

FIG. 17 is a schematic configuration view showing a system configuration of a radioactive isotope refining system related to a modified example.

FIG. 18 is a schematic configuration view showing an example of the radioactive isotope refining system in a case where ⁶⁴Cu is refined.

FIG. 19 is a schematic configuration view showing an example of the radioactive isotope refining system in a case where ⁸⁹Zr is refined.

FIG. 20 is a schematic configuration view showing an example of the radioactive isotope refining system in a case where ^{99m}Tc is refined.

### DETAILED DESCRIPTION

In a case where such a synthesizing apparatus is used for an examination or the like, it is desired to change flow channels freely. However, in the synthesizing apparatus described in the related art, a disposable module is exclusively used for synthesis for one type of drug. For this reason, since a user cannot change the flow channels of the disposable module freely, the disposable module cannot be used for synthesis of other types of drugs. The same problem occurs also in, for example, radioactive isotope handling apparatuses, such as a refining apparatus that refines a radioactive isotope.

It is desirable to provide a cassette for a radioactive isotope handling apparatus capable of handling a plurality of types of radioactive isotopes using one substrate, a radioactive isotope handling apparatuses, and a radioactive isotope handling system.

In the cassette for a radioactive isotope handling apparatus, a desired flow channel is formed by attaching the piping to some of the plurality of first holders and the plurality of second holders. For this reason, it is possible to form flow channels for handling desired radioactive isotopes using one substrate. As a result, it is possible to handle a plurality of types of radioactive isotopes using one substrate. Additionally, as at least two holders are provided corresponding to the through hole, the piping attached by the holders can be aligned above and with the through holes, and a structure capable of opening and closing the piping can be provided.

Each of the plurality of first holders may be provided on any of a plurality of first lines along the first direction, each of the plurality of second holders is provided on any of a plurality of second lines along the second direction, and each of the plurality of through holes may be provided so as to be aligned with an intersection point between any of the plurality of first lines and any of the plurality of second lines. In this case, the piping can be attached along the first lines and the second lines. Additionally, the attached piping can be more reliably aligned above and with the through holes, and a structure capable of more reliably opening and closing the piping can be provided.

At least three of the holders may be provided corresponding to each of the through holes. In this case, the degree of freedom in the attachment of the piping is further improved.

The through holes may be constituted by an elongated hole that extends along the second direction, and a plurality of the elongated holes may be provided at predetermined intervals along the first direction. In this case, since the through holes extend along the second direction and are formed in a wide range, the alignment between the piping and the through holes becomes easy.

In the radioactive isotope handling apparatus, a desired flow channel is formed by attaching the piping to some of the plurality of first holders and the plurality of second holders, using the cassette for a radioactive isotope handling apparatus fixed to the fixing portion. For this reason, it is possible to form flow channels for handling desired radioactive isotopes using one substrate. As a result, it is possible to handle a plurality of types of radioactive isotopes using one substrate. Additionally, the piping can be closed by pressing the piping against the through holes by the pressing members. For this reason, it is possible to open and close the piping.

In the radioactive isotope handling apparatus, the fixing portion may have a front surface that receives the substrate of the cassette for a radioactive isotope handling apparatus, a claw portion that supports the edge of the substrate, and a plurality of protruding portions that protrude from the front surface. The fixing portion can receive the substrate in the front surface, can support the edge of the substrate with the claw portions, and can make the protruding portions inserted through the through holes of the substrate. This enables the fixing portion to fix the cassette for a radioactive isotope handling apparatus reliably.

The radioactive isotope handling apparatus may further include a main body portion to which the cassette for a radioactive isotope handling apparatus is attached; and a door portion openably and closably provided at the main body portion. Additionally, the plurality of pressing members may be provided at the door portion, and may be capable of pressing the piping in a state where the door portion is closed. In this case, the positions of the pressing members can be changed in a state where the door portion is opened and a state where the door portion is closed. That is, in a case where the cassette for a radioactive isotope handling apparatus is attached and the radioactive isotope handling apparatus is operated and a case where the cassette for a radioactive isotope handling apparatus is removed, the positions of the pressing members can be changed, and it is possible to improve the workability of the attachment and removal of the cassette for a radioactive isotope handling apparatus.

In the radioactive isotope handling apparatus, the main body portion may include protruding portions at positions corresponding to the through holes provided in the cassette for a radioactive isotope handling apparatus, and the pressing members may be provided at positions corresponding to the protruding portions. By virtue of such a configuration, the pressing members can pinch the piping of the cassette between the pressing members and the protruding portions. This enables the pressing members to reliably block the piping, and enables the flow channels to be reliably set.

In a case where a portion that switches the direction of flow is present in the flow channel downstream of the extraction section, an inexpensive disposable three-way stopcock is used for a portion through which a liquid passes, and the drive section that is separate from the three-way stopcock is used for a portion that applies a driving force to the three-way stopcock. Thereby, in a case where different types of radioactive isotopes are refined, the drive section can be used as a common part irrespective of the type of radioactive isotopes, and the portion through which a liquid passes can be replaced with a new three-way stopcock. This can prevent degradation of refining performance with inexpensive structure.

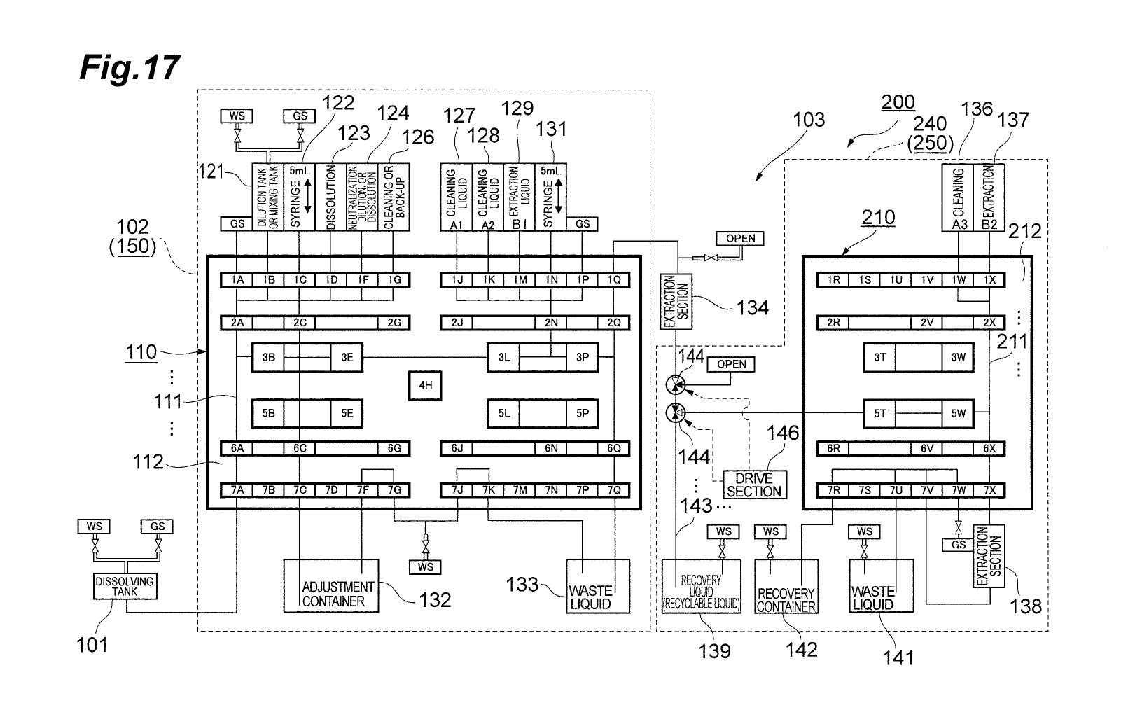

Embodiments of the invention will be described below in detail with reference to the accompanying drawings. In addition, in the following description, the same reference numerals will be given to the same elements, and the overlapping description will be omitted. Here, examples of the radioactive isotope handling apparatus includes a radioactive drug synthesizing apparatus that synthesizes a radioactive drug using radioactive isotopes, a solution adjusting apparatus that performs concentration adjustment of a solution containing radioactive isotopes, a radioactive isotope refining apparatus that refines radioactive isotopes, and the like. An example in a case where the radioactive drug synthesizing apparatus is adopted as the radioactive isotope handling apparatus will be described in one embodiment, an example in a case where the solution adjusting apparatus (in addition, the radioactive isotope refining apparatus is also illustrated in examples of FIGS. 17, 19, and 20) is adopted as the radioactive isotope handling apparatus will be described in another embodiment.

### One Embodiment

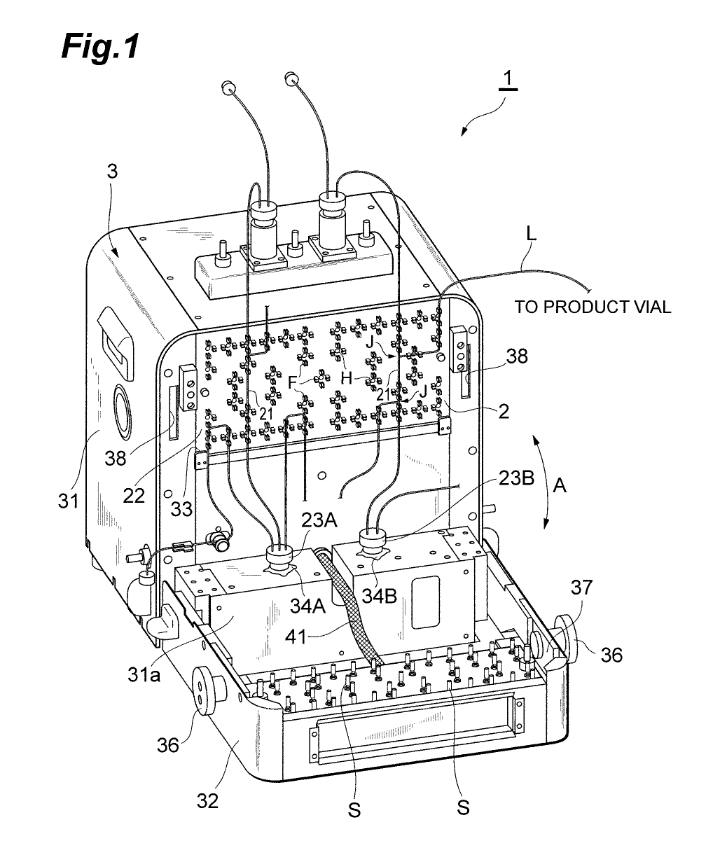

FIG. 1 is a perspective view showing the configuration of the radioactive drug synthesizing apparatus (hereinafter simply referred to as a “synthesizing apparatus”) related to the present embodiment. As shown in FIG. 1, the synthesizing apparatus 1 includes a detachable module 2 (a cassette for a radioactive isotope handling apparatus), and a fixed module (radioactive isotope handling apparatus) 3. In addition, the synthesizing apparatus 1 in the present embodiment functions as a radioactive drug synthesizing unit including the detachable module 2 corresponding to the cassette for a radioactive isotope handling apparatus, and the fixed module 3 corresponding to the radioactive isotope handling apparatus. In the following description, the up and down, front and rear, and left and right of the synthesizing apparatus 1 means the orientation when installation surface side in a case where the synthesizing apparatus 1 is installed is defined as down, and a side surface to which the detachable module 2 is attached is defined as front.

The detachable module 2 is a disposable cassette including a flow channel corresponding to a radioactive drug, and has piping 21, a plate 22 (substrate), and a reactor 23. The piping 21 is constituted by, for example, a silicone tube or the like, and forms flow channels for allowing a fluid to flow therethrough. The piping 21 is formed as a plurality of piping portions L are connected by a plurality of joint portions J. The joint portions J connect end portions of two or more piping portions L to each other. Additionally, the piping 21 has the joint portions J for connection with a reagent vial or the like.

The plate 22 is, for example, a substantially rectangular substrate for a radioactive drug synthesizing apparatus made of resin materials, such as polypropylene. The plate 22 has a plurality of strut portions F (holding means) capable of attaching the piping 21, and positions and holds the above-described piping 21 using some of the plurality of strut portions F at predetermined positions. A plurality of through holes H for opening and closing the piping 21 are provided at positions that face cylinders S (pressing members) to be described below, in the plate 22. The plate 22 will be described below in detail.

The reactor 23 is a vial that makes a raw material react to synthesize a radioactive drug containing a labeled compound.

The reactor 23 has a capacity of about 7 cc, has a flat bottom, a circular bottom, or a weight-shaped form, and has enhanced reactivity. The detachable module 2 may have a plurality of reactors 23 (reactors 23A and 23B) depending on a drug to be synthesized.

The fixed module 3 is a member with a substantially cubical appearance, and has a main body portion 31 and a door portion 32. A front surface of the main body portion 31 is provided with an attaching portion 33 that attaches the plate 22 of the detachable module 2. Moreover, a top surface of a step portion 31a the main body portion 31 is provided with an accommodating hole 34 (34A, 34B) that accommodates the reactor 23 (23A, 23B). A cooler for cooling the reactor 23, a heater for heating the reactor 23, a pressure sensor for checking the pressure within the reactor 23, a thermometer for checking the temperature within the reactor 23, a radiation sensor for checking the dose of radiation contained within the reactor 23, and the like are provided around the accommodating hole 34. Moreover, the main body portion 31 is provided with an electric furnace for heating the gas online, a mass flow controller that controls the flow rate of gas, an accommodating portion that detachably accommodates a reagent vial, and the like.

The door portion 32 is provided so as to be openable and closable in the direction of arrow A by 90 degrees via hinges provided in side surfaces of the step portion 31a of the main body portion 31. The door portion 32 can be opened and closed with respect to the main body portion 31 by rotating a knob 36 to engage and disengage a fixture 37 with/from engaging holes 38 of the main body portion 31. A plurality of cylinders S are provided at predetermined positions inside the door portion 32. The plurality of cylinders S are, for example, air cylinders that moves back and forth with the force of air. Air is supplied to the individual cylinders S via an air tube 41 that extends from the main body portion 31. Additionally, a back plate that protrudes toward the through holes H is provided at a position that faces the through holes H, in the main body portion 31. Thus, the cylinders S are enabled to press the piping 21 in a state where the door portion 32 is closed. By moving the cylinders S back and forth, pushing the piping 21 into the substantially circular through holes H, and sandwiching the piping 21 with the back plate and the cylinders S, the cylinders can be made to function as opening and closing valves VP that crush or restore the piping 21.

In such a synthesizing apparatus 1, the piping portions L that supply a refined drug solution extends toward a product vial that is not shown.

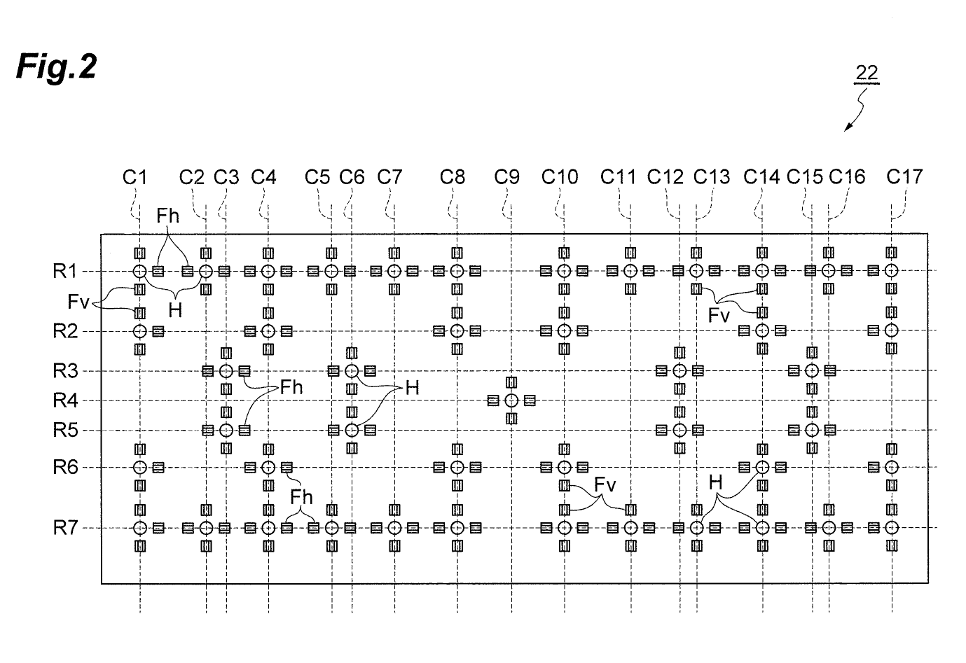

Subsequently, the above-described plate 22 will be described below in detail. FIG. 2 is a front view showing a configuration example of the plate 22 that the synthesizing apparatus 1 has. In the plate 22, the plurality of strut portions F includes a plurality of first strut portions Fv (first holding means) capable of attaching the piping 21 along an up-and-down direction (first direction), and a plurality of second strut portions Fh (second holding means) capable of attaching piping 21 along the left-and-right direction (second direction). Each of the first strut portions Fv is arranged on any line C of lines C1 to C17 (first lines) along the up-and-down direction. Additionally, each of the second strut portions Fh is arranged on any line R of lines R1 to R7 (second lines) along the left-and-right direction. In addition, the lines C1 to C17 are arranged in order from the left toward the right, and the lines R1 to R7 are arranged in order from the top toward the bottom.

Each of the through holes H is provided so as to be aligned with an intersection point between any of the lines C1 to C17 and any of the lines R1 to R7. In this example, the through holes H are provided so as to be aligned with intersection points between the a line R1 and lines C1, C2, C4, C5, C7, C8, C10, C11, C13, C14, C16, and C17, intersection points between a line R2 and lines C1, C4, C8, C10, C14, and C17, intersection points between a line R3 and lines C3, C6, C12, and C15, an intersection point between a line R4 and a line C9, intersection points between a line R5 and lines C3, C6, C12, and C15, intersection points between a line R6 and lines C1, C4, C8, C10, C14, and C17, and intersection points between a line R7 and lines C1, C2, C4, C5, C7, C8, C10, C11, C13, C14, C16, and C17, respectively.

Additionally, although at least two strut portions F are arranged to correspond to each of the through holes H, in this example, at least three strut portions F are arranged to correspond to each through hole. For example, three strut portions F are arranged corresponding to the peripheral edge of each through hole H provided on the leftmost line C1 among the lines C1 to C17. Specifically, the first strut portions Fv are provided above and below the through hole H, respectively and, the second strut portion Fh is provided on the right of the through hole H. Additionally, three strut portions F are arranged corresponding to the peripheral edge of each through hole H provided on the rightmost line C17 among the lines C1 to C17. Specifically, the first strut portions Fv are provided above and below the through hole H, respectively and, the second strut portion Fh is provided on the left of the through hole H. Additionally, four strut portions F are arranged corresponding to the peripheral edge of each through hole H provided on the lines C2 to C16. Specifically, the first strut portions Fv are provided above and below the through hole H, respectively and, the second strut portions Fh are provided on the left and right of the through hole H, respectively. In addition, the strut portions F may be provided except the peripheral edges of the through holes H. Additionally, the first strut portions Fv are provided on the lines C1 to C17 in portions sandwiched by the adjacent lines R1 to R7, and portions sandwiched by the through holes H located at both ends on the lines C1 to C17 and the end portions of the plate 22.

Next, an assembling method of the detachable module 2 using the plate 22 configured in this way will be described. First, in order to form a flow channel according to a radioactive drug that becomes a target to be synthesized, a combination of the strut portions F is selected. Then, the piping portions L are attached to the selected strut portions F, respectively. At this time, at least two strut portions F are selected with respect to one through hole H. For this reason, the piping portion L attached to the strut portion F is fixed so as to pass through the center of the through hole H. Subsequently, a plurality of piping portions L are connected to each other by the joint portion J. At this time, a T-shaped joint portion J or a Y-shaped joint portion J is used in a case where three piping portions L are connected, and a cross-shaped joint portion J is used in a case where four piping portions L are connected.

In this way, the piping 21 for synthesis of a radioactive drug, which has a flow channel according to the radioactive drug, is formed. Then, the reactor 23, a desired reagent vial, and the like are connected to the piping 21 via the joint portion J. The detachable module 2 is assembled as described above.

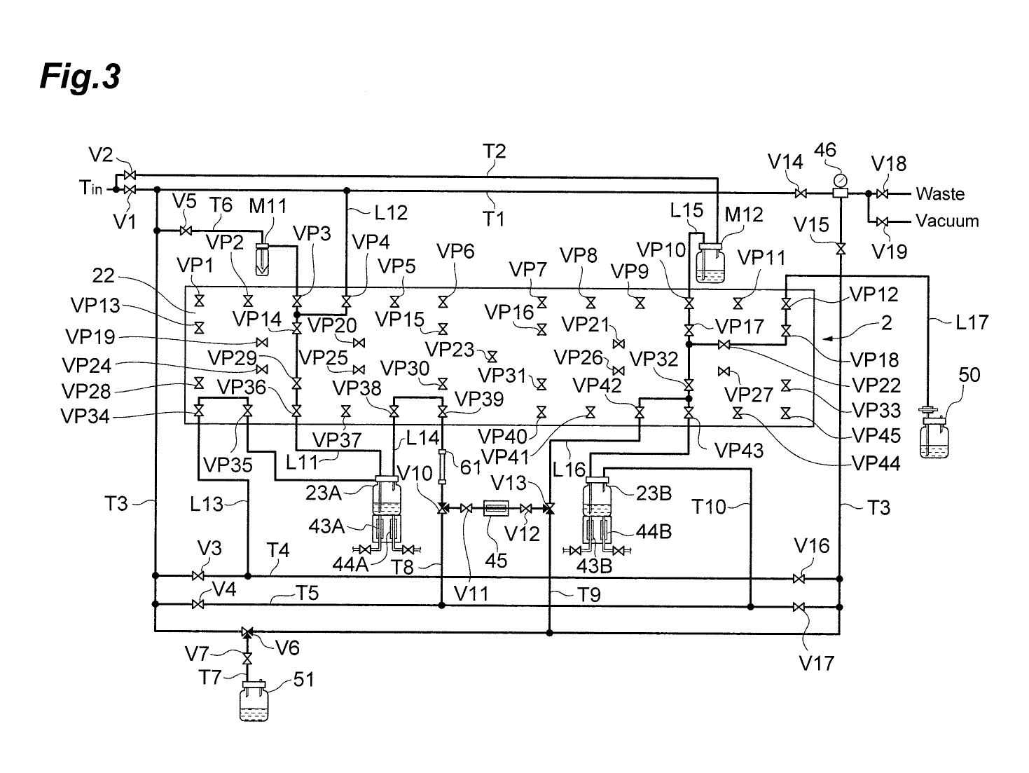

Configuration examples of the detachable module 2 according to a plurality of types of radioactive drugs, respectively, will be described below. In addition, in FIGS. 3 to 7, for convenience of description, opening and closing valves VP constituted by the through holes H and the cylinders S are shown instead of the through holes H, and the respective opening and closing valves VP is distinguished as an opening and closing valve VP1 to an opening and closing valve VP45 sequentially from the upper left. Additionally, connection of the piping is performed via the joint portions J or the like of shapes according to the number of pieces of piping to be connected, and the description thereof is omitted. Additionally, although the respective piping and piping portions may be constituted by one piping portion and may be constituted by a plurality of piping portions, symbols are given to predetermined ranges of piping and piping portion, respectively.

### First Configuration Example

In the first configuration example, a case where the detachable module 2 is used for an ¹¹C-methylation reaction will be described. FIG. 3 is a view showing the first configuration example of the detachable module 2 and the fixed module 3. As shown in FIG. 3, the fixed module 3 is provided with piping T, a cooler 43 (43A, 43B), a heater 44 (44A, 44B), an electric furnace 45, a product vial 50, a vial 51, a vial M (M11, M12), or the like. The piping T first provided in the fixed module 3 will be specifically described.

One end of piping T1 and one end of piping T2 are connected to piping Tin connected to an accelerator, and a bomb that supplies inert gas, which are not shown, by the T-shaped joint portion J. An opening and closing valve V1 is provided on one end side of the piping T1, and an opening and closing valve V14 is provided on the other end side thereof. The other end of the piping T1 is connected to a pressure gauge 46. The piping T2 extends to a vial M12, and an opening and closing valve V2 is provided in the path of the piping T2.

One end of piping T3 is connected between the opening and closing valve V1 and the opening and closing valve V14 of the piping T1. An opening and closing valve V6 and an opening and closing valve V15 are provided in the path of the piping T3, and the other end of the piping T3 is connected to the pressure gauge 46. Piping T4 is provided in order to bypass a portion in which the opening and closing valve V6 of the piping T3 is provided, and piping T5 is provided so as to further bypass the portion of the piping T3 bypassed by the piping T4. An opening and closing valve V3 is provided on one end side of the piping T4, and an opening and closing valve V16 is provided on the other end side thereof.

Additionally, an opening and closing valve V4 is provided on one end side of the piping T5, and an opening and closing valve V17 is provided on the other end side thereof. One end of piping T8 is connected between the opening and closing valve V4 and the opening and closing valve V17 of the piping T5. An opening and closing valve V10 is provided at the other end of the piping T8. Moreover, one end of piping T9 is connected to the portion of the piping T3 by passed by the piping TS. An opening and closing valve V13 is provided at the other end of the piping T9. The electric furnace 45 is provided via opening and closing valves V11 and V12 between the opening and closing valve V10 and the opening and closing valve V13.

Moreover, piping T6 is connected to one end side of the piping T3, and the piping T6 extends to a vial M11. An opening and closing valve V5 is provided in the path of the piping T6. Additionally, piping T7 is connected to the piping T3 via the opening and closing valve V6, and the piping T7 extends to a vial 51. An opening and closing valve V7 is provided in the path of the piping T7. A waste line and a vacuum line are connected to the pressure gauge 46. The waste line is a line that is evacuated at about −40 kPa. The vacuum line is a line that is evacuated at about −98 kPa. The waste line is provided with an opening and closing valve V18, and the vacuum line is provided with an opening and closing valve V19.

The above-described vial M11 is charged with, for example, about 0.5 mL of hydroiodic acid (HI). The vial M12 is charged with a solution containing sodium hydroxide.

Next, the detachable module 2 will be described. The detachable module 2 includes the piping 21, the plate 22, a reactor 23A, and a reactor 23B. The piping 21 is constituted by piping portions L11 to L17. The piping portion L11 extends from the vial M11 to the reactor 23A, and is attached so as to pass through the opening and closing valves VP3, VP14, VP29, and VP36 in order.

Here, attaching the piping portion L so as to pass through the opening and closing valve VP means that the piping portion L is fixed by at least two strut portions F provided at the peripheral edge of the opening and closing valve VP so as to pass above the through hole H. Additionally, in the drawings, the piping portion L that passes through the opening and closing valve VP in the up-and-down direction means the piping portion L fixed by two first strut portions Fv provided above and below the through hole H so as to pass above the through hole H, and the piping portion L that passes through the opening and closing valve VP in the left-and-right direction means the piping portion L fixed by two second strut portions Fh provided on the left and right of the through hole H so as to pass above the through hole H.

The piping portion L12 extends from between the opening and closing valve V1 and the opening and closing valve V14 of the piping T1 to between the opening and closing valve VP3 and the opening and closing valve VP14 of the piping portion L11, and is attached so as to pass through the opening and closing valve VP4. The piping portion L13 extends from between the opening and closing valve V3 and the opening and closing valve V16 of the piping T4 to the reactor 23A, and is attached so as to pass through the opening and closing valves VP34 and VP35 in order. The piping portion L14 extends from the reactor 23A to the opening and closing valve V10, and is attached so as to pass through the opening and closing valves VP38 and VP39 in order. Additionally, a filter 61 may be provided in the path of the piping portion L14.

The piping portion L15 extends from the vial M12 to the reactor 23B, and is attached so as to pass through the opening and closing valves VP10, VP17, VP32, and VP43 in order. The piping portion L16 extends from between the opening and closing valve VP32 and the opening and closing valve VP43 of the piping portion L15 to the opening and closing valve V13, and is provided so as to pass through the opening and closing valve VP42. The piping portion L17 extends from between the opening and closing valve VP17 and the opening and closing valve VP32 of the piping portion L15 to the product vial 50, and is attached so as to pass through the opening and closing valves VP22, VP18, and VP12 in order. Moreover, piping T10 is provided to extend from the reactor 23B to between the opening and closing valve V4 and the opening and closing valve V17 of the piping T5.

Next, the ¹¹C-methylation reaction using the detachable module 2 and the fixed module 3 will be described. First, the opening and closing valves VP1 to VP45 are closed. Then, the reactor 23A is charged with lithium aluminum hydride dissolved in THF (tetrahydrofuran), and is cooled to about −10° C. by a cooler 43A. Next, ¹¹CO₂ gas and lithium aluminum hydride are made to react by opening the opening and closing valves V1, V15, V16, V18, VP4, VP14, VP29, and VP34 to VP36, and blowing ¹¹CO₂ gas produced by the accelerator into the THF solution within the reactor 23A.

Thereafter, the opening and closing valves V15, V16, V18, VP34, and VP35 are opened, and the other opening and closing valves VP are closed. Then, the reactor 23A is decompressed by the waste line, and the reactor 23A is heated by a heater 44A. Thereby, the THF within the reactor 23A is evaporated and discharged, and the inside of the reactor 23A is dried.

Next, the opening and closing valves V1, V5, V15, V16, V18, VP3, VP14, VP29, and VP34 to VP36 are opened, and the other opening and closing valves are closed. Then, hydroiodic acid is introduced into the dried reactor 23A from the vial M11, and is made to perform an oxidization reaction with a salt containing ¹¹C generated in the above reaction. At this time, the hydroiodic acid within the vial M11 is transported to the reactor 23A by blowing inert gas into the vial M11 from the piping Tin. Although the solution or the like within the vial M may be transported by the same method, the description thereof is omitted. This synthesizes a ¹¹C-methyl iodide gas within the reactor 23A. The ¹¹C-methyl iodide gas synthesized here is handled as a basic reagent in the synthesis of a radioactive drug containing ¹¹C.

For example, the synthesis procedure of ¹¹C-methionine using the ¹¹C-methyl iodide gas will be described. The synthesis of ¹¹C-methionine is continuously performed using the detachable module 2. In addition, since the electric furnace 45 is not used, instead of the electric furnace 45 and the opening and closing valves V11 and V12, the opening and closing valve V10 and the opening and closing valve V13 are connected by piping.

First, the opening and closing valves VP1 to VP45 are closed. Then, homocysteine thiolactone that is a raw material of methionine is dissolved in acetone, and is charged into the reactor 23B. Thereafter, the opening and closing valves V1, V10, V13, V15, V17, V18, VP4, VP14, VP29, VP36, VP38, VP39, VP42, and VP43 are opened, and the ¹¹C-methyliodide gas synthesized in the reactor 23A is blown into the reactor 23B. This makes the ¹¹C-methyl iodide gas and the homocysteine thiolactone react.

Next, the opening and closing valves V2, V15, V17, V18, VP10, VP17, VP32, and VP43 are opened, and the other opening and closing valves are closed. Then, the solution containing sodium hydroxide within the vial M12 is introduced into the reactor 23B, and the compound within the reactor 23B is hydrolyzed. Subsequently, the opening and closing valves V1, V4, VP12, VP18, VP22, VP32, and VP43 are opened, and the other opening and closing valves are closed. Then, a reaction liquid within the reactor 23B is taken out, and is subjected to suitable refining treatment, and ¹¹C-methionine is recovered in the product vial 50 as a product drug.

Additionally, ¹¹C-raclopride and ¹¹C-flumazenil can also be synthesized using the ¹¹C-methyl iodide gas. The synthesis of ¹¹C-raclopride and ¹¹C-flumazenil is continuously performed using the detachable module 2. Since the synthesis procedures of these drugs are almost the same, a case where ¹¹C-raclopride is synthesized will be described. First, the opening and closing valves VP1 to VP45 are closed. Then, desmethyl raclopride that is a raw material of methyl iodide is dissolved in acetone containing sodium hydroxide in a minute amount, and is charged into the reactor 23B.

Thereafter, the opening and closing valves V1, V10 to V13, V15, V17, V18, VP4, VP14, VP29, VP36, VP38, VP39, VP42, and VP43 are opened, and the ¹¹C-methyl iodide gas synthesized in the reactor 23A is made to pass through the electric furnace 45 that is charged with silver trifluoromethanesulfonate (AgOTf). This makes the ¹¹C-methyl iodide gas and the silver trifluoromethanesulfonate react online, to synthesize a ¹¹C-methyl triflate gas.

Then, this ¹¹C-methyl triflate gas is blown into the reactor 23B, and the ¹¹C-methyl triflate gas and desmethyl raclopride are made to react. Next, the opening and closing valves V1, V4, VP12, VP18, VP22, VP32, and VP43 are opened, and the other opening and closing valves are closed. Then, the reaction liquid within the reactor 23B is taken out, and is subjected to suitable refining treatment, and ¹¹C-raclopride is recovered in the product vial 50 as a product drug.

### Second Configuration Example

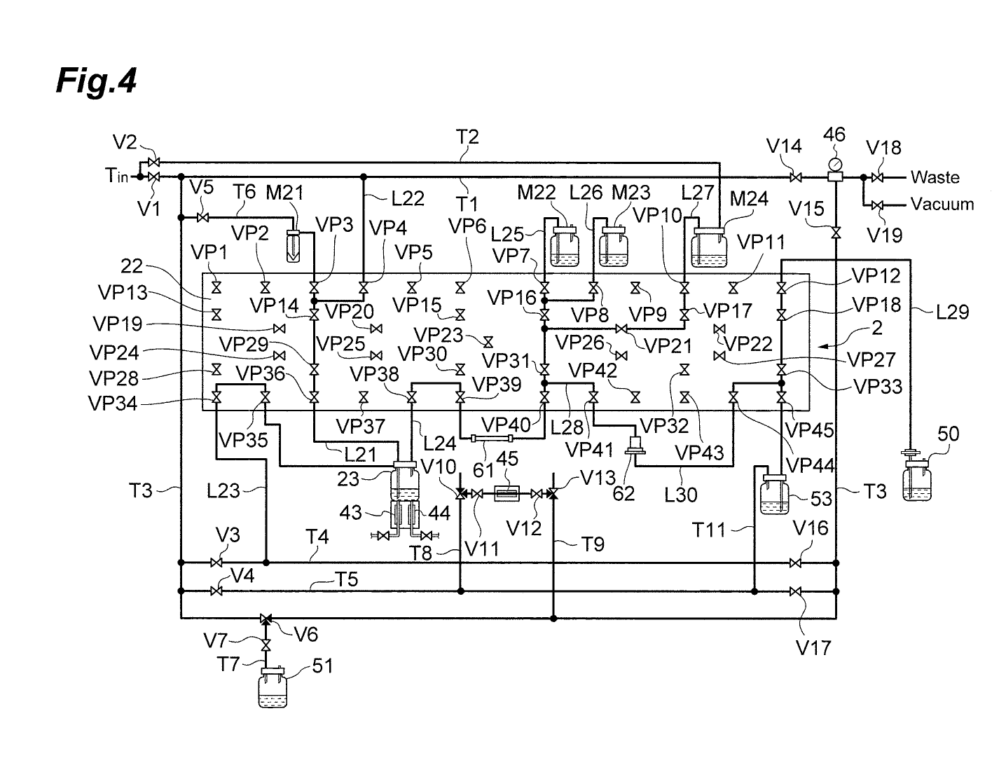

In the second configuration example, a case where the detachable module 2 is used for the synthesis of ¹¹C-choline will be described. FIG. 4 is a view showing the second configuration example of the detachable module 2 and the fixed module 3. As shown in FIG. 4, the fixed module 3 is different from the fixed module 3 of the first configuration example in that this fixed module includes a vial M21 and a vial M24 instead of the vial M11 and the vial M12, and further includes a vial M22, a vial M23, a waste liquid bottle 53, a filter 61, and a column 62.

The vial M21 is charged with about 0.5 mL of hydroiodic acid (HI). The vial M22 is charged with about 10 mL of ethanol/water. The vial M23 is charged with about 10 mL of water. The vial M24 is charged with about 10 mL of physiological salt solution. The column 62 is a tubular packed with resin. Since this fixed module is the same as the fixed module 3 of the first configuration example in the other configuration, the description thereof is omitted.

The detachable module 2 includes piping 21, the plate 22, and the reactor 23. The piping 21 is constituted by piping portions L21 to L30. The piping portion L21 extends from the vial M21 to the reactor 23, and is attached so as to pass through the opening and closing valves VP3, VP14, VP29, and VP36 in order. The piping portion L22 extends from between the opening and closing valve V1 and the opening and closing valve V14 of the piping T1 to between the opening and closing valve VP3 and the opening and closing valve VP14 of the piping portion L21, and is attached so as to pass through the opening and closing valve VP4.

The piping portion L23 extends from between the opening and closing valve V3 and the opening and closing valve V16 of the piping T4 to the reactor 23, and is attached so as to pass through the opening and closing valves VP34 and VP35 in order. The piping portion L24 extends from the reactor 23 to one end of the filter 61, and is attached so as to pass through the opening and closing valve VP38 and VP39 in order. The piping portion L25 extends from the vial M22 to the other end of the filter 61, and is attached so as to pass through the opening and closing valves VP7, VP16, VP31, and VP40 in order. The piping portion L26 extends from the vial M23 to between the opening and closing valve VP7 and the opening and closing valve VP16 of the piping portion L25, and is provided so as to pass through the opening and closing valve VP8.

The piping portion L27 extends from the vial M24 to between the opening and closing valve VP16 and the opening and closing valve VP31 of the piping portion L25, and is provided so as to pass through the opening and closing valves VP10, VP17, and VP21 in order. The piping portion L28 extends from between the opening and closing valve VP31 and the opening and closing valve VP40 of the piping portion L25 to one end of the column 62, and is provided so as to pass through the opening and closing valve VP41.

The piping portion L29 extends from the waste liquid bottle 53 to the product vial 50, and is attached so as to pass through the opening and closing valves VP45, VP33, VP18, and VP12 in order. The piping portion L30 extends from between the opening and closing valve VP33 and the opening and closing valve VP45 of the piping portion L29 to the other end of the column 62, and is provided so as to pass through the opening and closing valve VP44. Moreover, piping T11 is provided to extend from the waste liquid bottle 53 to between the opening and closing valve V4 and the opening and closing valve V17 of the piping T5.

Next, the synthesis procedure of ¹¹C-choline using the detachable module 2 and the fixed module 3 will be described. As a previous stage of the synthesis of ¹¹C-choline, a ¹¹C-methyl iodide gas is synthesized in the reactor 23 by the detachable module 2 of FIG. 3. Next, ¹¹C-choline is synthesized using the detachable module 2 of FIG. 4. Specifically, first, the opening and closing valves VP1 to VP45 are closed. Then, the column 62 is charged with 2-dimethylamino ethanol that is a raw material of choline.

Thereafter, the opening and closing valves V1, V3, V15, V17, V18, VP34, VP35, VP38 to VP41, VP44, and VP45 are opened, and the ¹¹C-methyl iodide gas synthesized in the reactor 23 is made to pass through the column 62. This makes ¹¹C-methyl iodide gas and 2-dimethylamino ethanol react, to synthesize ¹¹C-choline.

Next, the opening and closing valves V15, V17, V18, VP7, VP16, VP31, VP41, VP44, and VP45 are opened, and the other opening and closing valves are closed. Then, the ethanol/water within the vial M22 is introduced into the column 62, and unreacted 2-dimethylamino ethanol within the column 62 is cleaned. Next, the opening and closing valves V2, VP10, VP12, VP17, VP18, VP21, VP31, VP33, VP41, and VP44 are opened, and the other opening and closing valves are closed. Then, the physiological salt solution within the vial M24 is introduced into the column 62, and ¹¹C-choline is recovered in the product vial 50 as a product drug.

### Third Configuration Example

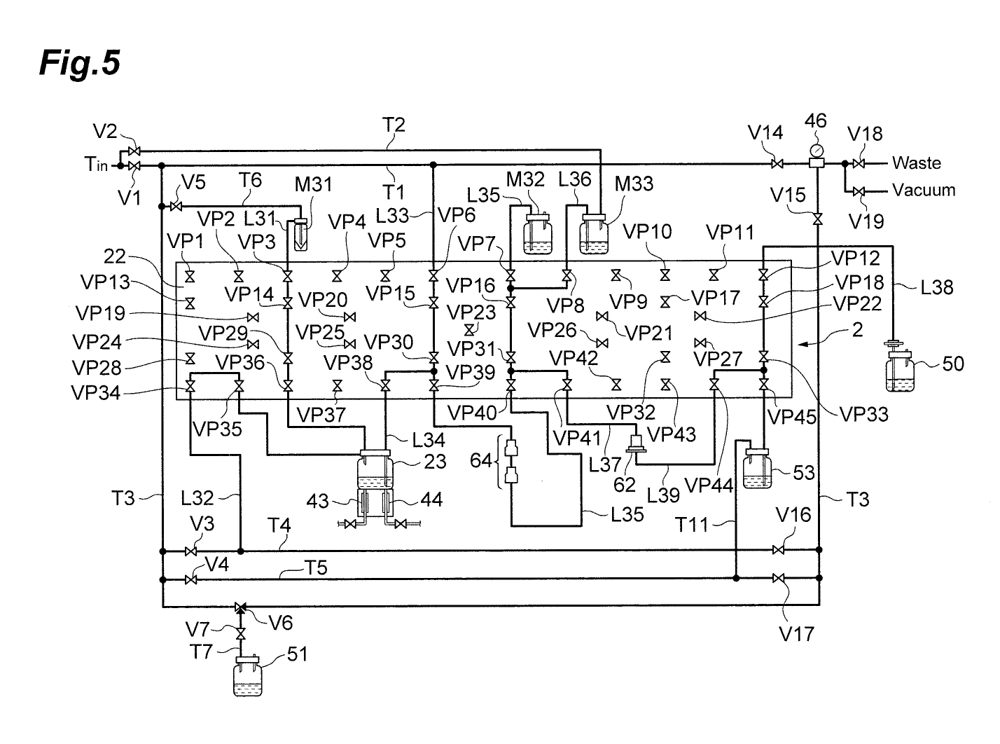

In the third configuration example, a case where the detachable module 2 is used for synthesis of ¹¹C-acetic acid will be descried. FIG. 5 is a view showing the third configuration example of the detachable module 2 and the fixed module 3. As shown in FIG. 5, the fixed module 3 is different from the fixed module 3 of the first configuration example in that this fixed module includes a vial M31 and a vial M33 instead of the vial M11 and the vial M12, and further includes a vial M32, the waste liquid bottle 53, the column 62, and a column 64. The vial M31 is charged with about 0.5 mL of hydrochloric acid (HCl) of 1 mol/L. The vial M32 is charged with about 10 mL of water. The vial M33 is charged with a physiological salt solution. The column 62 is a column that is charged with anionic exchange resin, and temporarily traps acetic acid contained in a passed liquid. Additionally, as the physiological salt solution is passed through the column 62 after the column 62 traps the acetic acid, ion exchange is performed within the column 62 again to extract the acetic acid. The column 64 is a column that is charged with a cationic exchange resin was charged, and exchanges magnesium ions contained in the reaction liquid with silver ions. Since this fixed module is the same as the fixed module 3 of the first configuration example in the other configuration, the description thereof is omitted.

The detachable module 2 includes the piping 21, the plate 22, and the reactor 23. The piping 21 is constituted by piping portions L31 to L39. The piping portion L31 extends from the vial M31 to the reactor 23, and is attached so as to pass through the opening and closing valves VP3, VP14, VP29, and VP36 in order. The piping portion L32 extends from between the opening and closing valve V3 and the opening and closing valve V16 of the piping T4 to the reactor 23, and is attached so as to pass through the opening and closing valves VP34 and VP35 in order. The piping portion L33 extends from between the opening and closing valve V1 and the opening and closing valve V14 of the piping T1 to one end of the column 64, and is attached so as to pass through the opening and closing valves VP6, VP15, VP30, and VP39 in order.

The piping portion L34 extends from between the opening and closing valve VP30 and the opening and closing valve VP39 of the piping portion L33 to the reactor 23, and is attached so as to pass through the opening and closing valve VP38. The piping portion L35 extends from the vial M32 to the other end of the column 64, and is attached so as to pass through the opening and closing valves VP7, VP16, VP31, and VP40 in order. The piping portion L36 extends from the vial M33 to between the opening and closing valve VP7 and the opening and closing valve VP16 of the piping portion L35, and is attached so as to pass through the opening and closing valve VP8. The piping portion L37 extends from between the opening and closing valve VP31 and the opening and closing valve VP40 of the piping portion L35 to one end of the column 62, and is attached so as to pass through the opening and closing valve VP41.

The piping portion L38 extends from the waste liquid bottle 53 to the product vial 50, and is attached so as to pass through the opening and closing valves VP45, VP33, VP18, and VP12 in order. The piping portion L39 extends from between the opening and closing valve VP33 and the opening and closing valve VP45 of the piping portion L38 to the other end of the column 62, and is attached so as to pass through the opening and closing valve VP44. Moreover, the piping T11 is attached to extend from the waste liquid bottle 53 to between the opening and closing valve V4 of the piping T5 and the opening and closing valve V17.

Next, the synthesis procedure of ¹¹C-acetic acid using the detachable module 2 and the fixed module 3 will be described. First, the opening and closing valves VP1 to VP45 are closed. Then, the reactor 23 is charged with a THF solution of methyl magnesium bromide that is a Grignard reagent. Thereafter, the opening and closing valves V1, V15, V16, V18, VP6, VP15, VP30, VP34, VP35, and VP38 are opened, and the ¹¹C—CO₂ gas produced by the accelerator is blown into the reactor 23. This makes the ¹¹C—CO₂ gas and the THF solution of methyl magnesium bromide react.

Next, the opening and closing valves V1, V5, V15, V16, V18, VP3, VP14, VP29, and VP34 to VP36 are opened, and the other opening and closing valves are closed. Then, the hydrochloric acid within the vial M31 is introduced into the reactor 23, the compound within the reactor 23 is hydrolyzed, and ¹¹C-acetic acid is synthesized within the reactor 23. Subsequently, the opening and closing valves V1, V3, VP12, VP18, VP33 to VP35, VP38 to VP41, and VP44 are opened, and the other opening and closing valves are closed. Then, the reaction liquid within the reactor 23 is taken out through the column 64 and the column 62, and is subjected to suitable refining treatment, and ¹¹C-acetic acid is recovered in the product vial 50 as a product drug.

### Fourth Configuration Example

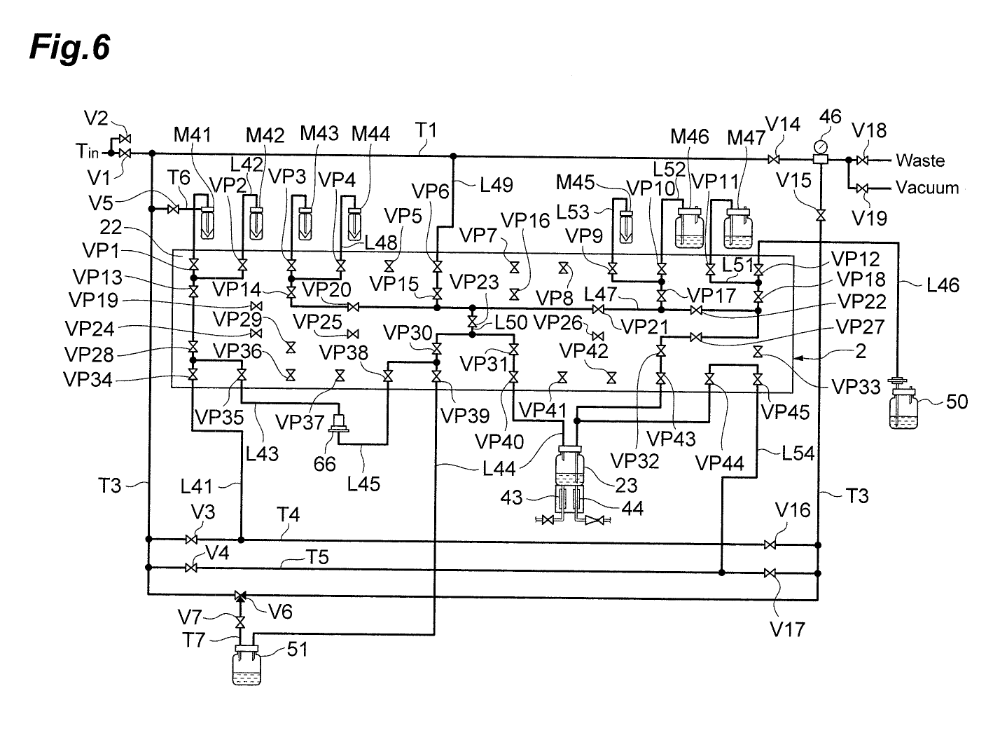

In a fourth configuration example, a case where the detachable module 2 is used for the synthesis of ¹⁸F-FDG, ¹⁸F-FLT, and ¹⁸F-FMISO will be described. FIG. 6 is a view showing the fourth configuration example of the detachable module 2 and the fixed module 3. As shown in FIG. 6, the fixed module 3 is different from the fixed module 3 of the first configuration example in that this fixed module includes a vial M41 instead of the vial M11, and further includes a vial M42 to a vial M47, and ion exchange resin 66. Since the piping T2 is not used in this configuration example, it is not necessary to provide this piping.

The vial M41 temporarily stores water containing ¹⁸F ions produced by the accelerator. The vial M42 is charged with a solution containing about 0.7 mL of phase transfer catalyst (K.222) and containing about 0.2 mL of potassium carbonate aqueous solution. The vial M43 is charged with about 0.5 mL of acetonitrile (MeCN). The vial M44 is charged with about 1.5 mL of acetonitrile in which about 20 mg of trifluoromethanesulfonyl mannopyranose is dissolved if the main raw material of a synthetic drug is, for example, FDG.

The vial M45 is charged with 1 mol/L of hydrochloric acid or about 0.75 mL of sodium hydroxide. The vials M46 and M47 are charged with drugs for performing required reaction treatment added by a drug to be synthesized. A physiological salt solution is charged, for example, in the case of FDG, and a phosphate buffer solution or the like for adjusting pH is changed, for example, in the case of FLT. The ion exchange resin 66 is resin that exchanges ionic substance within resin with ionic substance in an electrolyte bypassing through an electrolyte. Since this fixed module is the same as the fixed module 3 of the first configuration example in the other configuration, the description thereof is omitted.

The detachable module 2 includes the piping 21, the plate 22, and the reactor 23. The piping 21 is constituted by piping portions L41 to L54. The piping portion L41 extends from the vial M41 to between the opening and closing valve VP3 and the opening and closing valve VP16 of the piping T4, and is attached so as to pass through the opening and closing valves VP1, VP13, VP28, and VP34 in order. The piping portion L42 extends from the vial M42 to between the opening and closing valve VP1 and opening and closing valve VP13 of the piping portion L41, and is attached so as to pass through the opening and closing valve VP2.

The piping portion L43 extends from between the opening and closing valve VP28 and the opening and closing valve VP34 of the piping portion L41 to one end of the ion exchange resin 66, and is attached so as to pass through the opening and closing valve VP35. The piping portion L44 extends from the vial 51 to the reactor 23, and is attached so as to pass through the opening and closing valves VP39, VP30, VP31, and VP40 in order. The piping portion L45 extends from between the opening and closing valve VP30 and the opening and closing valve VP39 of the piping portion L44 to the other end of the ion exchange resin 66, and is attached so as to pass through the opening and closing valve VP38.

The piping portion L46 extends from the reactor 23 to the product vial 50, and is attached so as to pass through the opening and closing valve VP43, VP32, VP27, VP18, and VP12 in order. The piping portion L47 extends from the vial M43 to between opening and closing valve VP18 and the opening and closing valve VP27 of the piping portion L46, and is attached so as to pass through the opening and closing valves VP3, VP14, VP20, VP21, and VP22 in order. The piping portion L48 extends from the vial M44 to between the opening and closing valve VP3 and the opening and closing valve VP14 of the piping portion L47, and is attached so as to pass through the opening and closing valve VP4.

The piping portion L49 extends from between opening and closing valve V1 and the opening and closing valves V14 of the piping T1 to between the opening and closing valve VP20 and the opening and closing valve VP21 of the piping portion L47, and is attached so as to pass through the opening and closing valves VP6 and VP15 in order. The piping portion L50 extends from between the opening and closing valve VP20 and the opening and closing valve VP21 of the piping portion L47 to between the opening and closing valve VP30 and the opening and closing valve VP31 of the piping portion L44, and is attached so as to pass through the opening and closing valve VP23. The piping portion L51 extends from the vial M47 to between the opening and closing valve VP12 and the opening and closing valve VP18 of the piping portion L46, and is attached so as to pass through the opening and closing valve VP11.

The piping portion L52 extends from the vial M46 to between the opening and closing valve VP21 and the opening and closing valve VP22 of the piping portion L47, and is attached so as to pass through the opening and closing valves VP10 and VP17 in order. The piping portion L53 extends from the vial M45 to between the opening and closing valve VP10 and the opening and closing valve VP17 of the piping portion L52, and is attached so as to pass through the opening and closing valve VP9. The piping portion L54 extends from between the opening and closing valve VP43 of the piping portion L46 and the reactors 23 to between the opening and closing valve V4 and the opening and closing valve V17 of the piping T5, and is attached so as to pass through the opening and closing valves VP44 and VP45 in order.

Next, the synthesis procedure of ¹⁸F-FDG, ¹⁸F-FLT, and ¹⁸F-FMISO using the detachable module 2 will be described. Since the synthesis procedure of these drugs is almost the same, a case where ¹⁸F-FDG is synthesized will be described here. First, the opening and closing valves VP1 to VP45 are closed. Then, the opening and closing valves V1, V5, VP1, VP13, VP28, VP35, VP38, and VP39 are opened, and water containing F ions produced i by the accelerator is once stored in the vial M41. Thereafter, the water containing ¹⁸F ions is passed through the ion exchange resin 66, and the ¹⁸F ions are made to be adsorbed on the ion exchange resin 66.

Next, the opening and closing valves VP2, VP13, VP28, VP30, VP31, VP35, VP38, and VP40 are opened, and the other opening and closing valves are closed. Then, a potassium carbonate aqueous solution mixed with a phase transfer catalyst is passed through the ion exchange resin 66 from the vial M42, and the ¹⁸F ions adsorbed on the ion exchange resin 66 are transported to the reactor 23 together with the phase transfer catalyst. By mixing this phase transfer catalyst with an electrolyte (ions), it is possible to dissolve the electrolyte in an aprotic solvent, such as acetonitrile. In addition, the phase transfer catalyst may be introduced into the reactor 23 after the potassium carbonate aqueous solution is passed through the ion exchange resin and the ¹⁸F ions are transported to the reactor 23.

Subsequently, the opening and closing valves V15, V17, V18, VP44, and VP45 are opened, and the other opening and closing valves are closed. Then, the reactor 23 is heated by the heater 44 and the reactor 23 is decompressed by the waste line, to dry the inside of the reactor 23. Next, the opening and closing valves VP4, VP14, VP20, VP23, VP31, and VP40 are opened, and the other opening and closing valves are closed. Then, a dissolving liquid in which trifluoromethanesulfonyl mannopyranose is dissolved in acetonitrile is introduced into the reactor 23 from the vial M44. This causes a fluorination reaction of the ¹⁸F ions mixed with the phase transfer catalyst and the dissolving liquid.

Subsequently, the opening and closing valves VP9, VP17, VP22, VP27, VP32, and VP43 are opened, and the other opening and closing valves are closed. Then, the hydrochloric acid (or sodium hydroxide) within the vial M45 is introduced into the reactor 23, and the compound within the reactor 23 is hydrolyzed. Next, the opening and closing valves V1, VP6, VP12, VP15, VP18, VP23, VP27, VP31, VP32, VP40, and VP43 are opened, and the other opening and closing valves are closed. Then, the reaction liquid in a reactor 23 is taken out, and is subjected to suitable refining treatment, and ¹⁸F-FDG is recovered in the product vial 50 as a product drug.

### Fifth Configuration Example

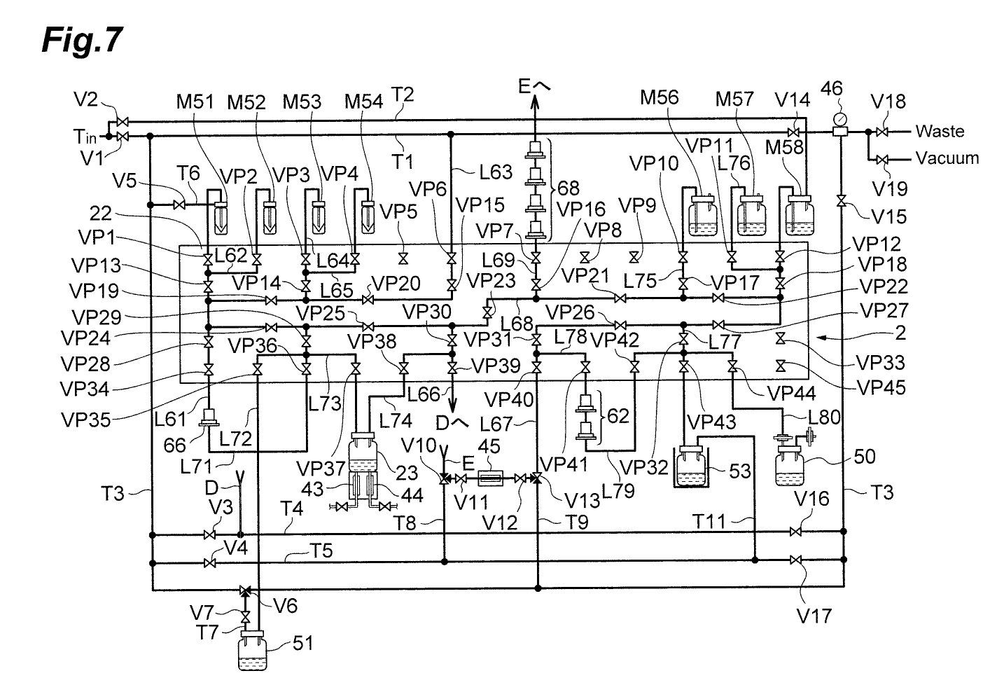

In the fifth configuration example, a case where the detachable module 2 is used for the synthesis of ¹⁸F—F-choline will be described. FIG. 7 is a view showing the fifth configuration example of the detachable module 2 and the fixed module 3. As shown in FIG. 7, the fixed module 3 is different from the fixed module 3 of the first configuration example in that this fixed module includes a vial M51 and a vial M58 instead of the vial M11 and the vial M12, and further includes a vial M52 to a vial M54, a vial M56, a vial M57, the waste liquid bottle 53, the column 62, the ion exchange resin 66, and a silica gel column 68.

The vial M51 temporarily stores water containing ¹⁸F ions produced by the accelerator. The vial M52 is charged with a solution containing about 0.7 mL of phase transfer catalyst (K.222) and containing about 0.2 mL of potassium carbonate aqueous solution. The vial M53 is charged with about 0.5 mL of acetonitrile. The vial M54 is charged with about 1 mL of acetonitrile in which about 200 μL of dibromomethane (CH₂Br₂) is dissolved.

The vial M56 is charged with about 10 mL of ethanol. The vial M57 is charged with about 10 mL of water. The vial M58 is charged with about 10 mL of physiological salt solution. The silica gel column 68, which is a tubular container packed with silica gel, makes a reaction object stagnate in a tube, and separates the reaction object from foreign matter. Since this fixed module is the same as the fixed module 3 of the first configuration example in the other configuration, the description thereof is omitted.

The detachable module 2 includes the piping 21, the plate 22, and the reactor 23. The piping 21 is constituted by piping portions L61 to L80. The piping portion L61 extends from the vial M51 to one end of the ion exchange resin 66, and is attached so as to pass through the opening and closing valves VP1, VP13, VP28, and VP34 in order. The piping portion L62 extends from the vial M52 to between the opening and closing valve VP1 and the opening and closing valve VP13 of the piping portion L61, and is attached so as to pass through the opening and closing valve VP2.

The piping portion L63 extends from between the opening and closing valve V1 and the opening and closing valves V14 of the piping T1 to between the opening and closing valve VP13 and the opening and closing valve VP28 of the piping portion L61, and is attached so as to pass through the opening and closing valves VP6, VP15, VP20, and VP19 in order. The piping portion L64 extends from the vial M53 to between the opening and closing valve VP19 and the opening and closing valve VP20 of the piping portion L63, and is attached so as to pass through the opening and closing valves VP3 and VP14 in order. The piping portion L65 extends from the vial M54 to between the opening and closing valve VP3 and the opening and closing valve VP14 of the piping portion L64, and is attached so as to pass through the opening and closing valve VP4.

The piping portion L66 extends from between the opening and closing valve VP23 and the opening and closing valve VP25 of the piping portion L68 to between the opening and closing valve V3 and the opening and closing valve V16 of the piping T4, and is attached so as to pass through the opening and closing valves VP30 and VP39 in order. The piping portion L67 extends from the vial M58 to the opening and closing valve V13, and is attached so as to pass through the opening and closing valves VP12, VP18, VP27, VP26, VP31, and VP40 in order. The piping portion L68 extends from between the opening and closing valve VP13 and the opening and closing valve VP28 of the piping portion L61 to between the opening and closing valve VP18 and the opening and closing valve VP27 of the piping portion L67, and is attached so as to pass through the opening and closing valves VP24, VP25, VP23, VP21, and VP22 in order.

The piping portion L69 extends from between the opening and closing valve VP21 and the opening and closing valve VP23 of the piping portion L68 to one end of the silica gel column 68, and is attached so as to pass through the opening and closing valves VP16 and VP7 in order. The other end of the silica gel column 68 is connected to the opening and closing valve V10. The piping portion L71 extends from between the opening and closing valve VP24 and the opening and closing valve VP25 of the piping portion L68 to the other end of the ion exchange resin 66, and is attached so as to pass through the opening and closing valves VP29 and VP36 in order. The piping portion L72 extends from between the opening and closing valve VP29 and the opening and closing valve VP36 to the piping portion L71 to the vial 51, and is attached so as to pass through the opening and closing valve VP35.

The piping portion L73 extends from between the opening and closing valve VP29 and the opening and closing valve VP36 of the piping portion L71 to the reactor 23, and is attached so as to pass through the opening and closing valve VP37. The piping portion L74 extends from between the opening and closing valve VP30 and the opening and closing valve VP39 of the piping portion L66 to the reactor 23, and is attached so as to pass through the opening and closing valve VP38. The piping portion L75 extends from the vial M56 to between the opening and closing valve VP21 and the opening and closing valve VP22 of the piping portion L68, and is attached so as to pass through the opening and closing valves VP10 and VP17 in order. The piping portion L76 extends from the vial M57 to between the opening and closing valve VP12 and the opening and closing valve VP18 of the piping portion L67, and is attached so as to pass through the opening and closing valve VP11.

The piping portion L77 extends from between the opening and closing valve VP26 and the opening and closing valve VP27 of the piping portion L67 to the waste liquid bottle 53, and is attached so as to pass through the opening and closing valves VP32 and VP43 in order. The piping portion L78 extends from between the opening and closing valve VP31 and the opening and closing valve VP40 of the piping portion L67 to one end of the column 62, and is attached so as to pass through the opening and closing valve VP41. The piping portion L79 extends from between the opening and closing valve VP32 and the opening and closing valve VP43 of the piping portion L77 to the other end of the column 62, and is attached so as to pass through the opening and closing valve VP42. The piping portion L80 extends from between the opening and closing valve VP32 and the opening and closing valve VP43 of the piping portion L77 to the product vial 50, and is attached so as to pass through the opening and closing valve VP44.

Next, the synthesis procedure of ¹⁸F—F-choline using the detachable module 2 will be described. First, the opening and closing valves VP1 to VP45 are closed. Then, the opening and closing valves V1, V5, VP1, VP13, VP28, and VP34 to VP36 are opened, and water containing ¹⁸F ions produced by the accelerator is once stored in the vial M51. Thereafter, the water containing ¹⁸F ions is passed through the ion exchange resin 66, and the ¹⁸F ions are made to be adsorbed on the ion exchange resin 66.

Next, the opening and closing valves VP2, VP13, VP28, VP34, VP36, and VP37 are opened, and the other opening and closing valves are closed. Then, a potassium carbonate aqueous solution mixed with a phase transfer catalyst is passed through the ion exchange resin 66 from the vial M52, and the ¹⁸F ions adsorbed on the ion exchange resin 66 are transported to the reactor 23 together with the phase transfer catalyst. In addition, the phase transfer catalyst may be separately introduced into the reactor 23 from the vial 53 after the potassium carbonate aqueous solution is passed through the ion exchange resin and the ¹⁸F ions are transported to the reactor 23. In this case, the opening and closing valves VP3, VP14, VP19, VP24, VP29, and VP37 are opened, and the other opening and closing valves are closed.

Subsequently, the opening and closing valves V15, V16, V19, VP38, and VP39 are opened, and the other opening and closing valves are closed. Then, the reactor 23 is heated by the heater 44 and the reactor 23 is decompressed by the waste line, to dry the inside of the reactor 23. Next, the opening and closing valves VP4, VP14, VP19, VP24, VP29, and VP37 are opened, and the other opening and closing valves are closed. Then, a dissolving liquid in which dibromomethane dissolved in acetonitrile is introduced into the reactor 23 from the vial M54. This causes a fluorination reaction of the ¹⁸F ions mixed with the phase transfer catalyst and the dissolving liquid, and synthesizes ¹⁸F-brominated methyl fluoride gas.

Subsequently, the opening and closing valves V1, V10, V15, V17, V18, VP6, VP7, VP15, VP16, VP19, VP20, VP23, VP24, VP29, VP30, VP37, and VP38 are opened, and the other opening and closing valves are closed. Then, the ¹⁸F-brominated methyl fluoride gas within the reactor 23 is passed through the silica gel column 68, and if the object (¹⁸F-brominated methyl fluoride gas) is checked, the object is separately taken. At this time, gas is wasted through a waste line until the object is checked. Next, the opening and closing valves V1, V10 to V13, VP6, VP7, VP15, VP16, VP19, VP20, VP23, VP24, VP29, VP30, VP37, VP38, VP40 to VP43 are opened, and the other opening and closing valves are closed. Then, ¹⁸F—F-choline is synthesized by passing the separately taken ¹⁸F-brominated methyl fluoride gas through the column 62 charged with 2-dimethylamino ethanol that is a raw material of choline.

Thereafter, the opening and closing valves VP10, VP17, VP22, VP26, VP27, VP31, and VP41 to VP43 are opened, and the other opening and closing valves are closed. Then, ethanol within the vial M56 is introduced into the column 62, and the unreacted 2-dimethylamino ethanol that remains within the column 62 is cleaned. Moreover, the opening and closing valves VP11, VP18, VP26, VP27, VP31, and VP41 to VP43 are opened, and the other opening and closing valves are closed. Then, the water within the vial M57 is introduced into the column 62, and the ethanol that remains within the column 62 is cleaned. Next, the opening and closing valves VP12, VP18, VP26, VP27, VP31, VP41, VP42, and VP44 are opened, and the other opening and closing valves are closed. Then, the physiological salt solution within the vial M58 is introduced into the column 62, and ¹⁸F—F-choline is recovered in the product vial 50 as a product drug.

As described above in detail, in the synthesizing apparatus 1, the piping 21 of various shapes can be attached using one plate 22, and a flow channel for synthesizing a desired radioactive drug can be formed using one plate. For this reason, a plurality of types of radioactive drugs can be synthesized using one plate 22 without preparing a plate only for a radioactive drug to be syntheses. Additionally, as at least two strut portions F are provided corresponding to the through hole H, the piping 21 attached by the strut portions F can be aligned with the through hole H, and a structure capable of opening and closing the piping 21 can be provided.

In addition, the cassette for a radioactive drug synthesizing apparatus, a radioactive drug synthesizing apparatus, and the substrate for a radioactive drug synthesizing apparatus related to the invention, are not limited to those described in the present embodiment. For example, in the plate 22, the respective positions of the plurality of through holes H may be appropriately changed.

Additionally, the lines C where the first strut portions Fv are arranged and the lines R where the second strut portions Fh are arranged are not limited to those described in the above embodiment. The number of the lines C and the number of the lines R may be changed if needed. For example, the respective lines C and the respective lines R may intersect each other in nine or more locations. Additionally, the extending direction of the lines C and the extending direction of the lines R are not limited to the up-and-down direction and the left-and-right direction, but the lines C and the lines R only need to intersect each other.

Additionally, although the strut portions F are arranged above and below and on the left and right of the through hole H, the strut portions F may be arranged in other directions of the peripheral edge portion of the through hole H. Two or more strut portions F only need to be provided with respect to the through hole H, and the directions in which the strut portions F are provided are not limited. Additionally, in the above embodiment, the strut portions F are not provided at end portions of the plate 22 in the left-and-right direction. However, the strut portions F may be provided if needed.

Additionally, other holding means capable of attaching the piping portion L and capable of fixing the position of the piping portion L may be provided instead of the strut portions F. For example, instead of the strut portions F, grooves may be provided in the plate 22.

### Another Embodiment

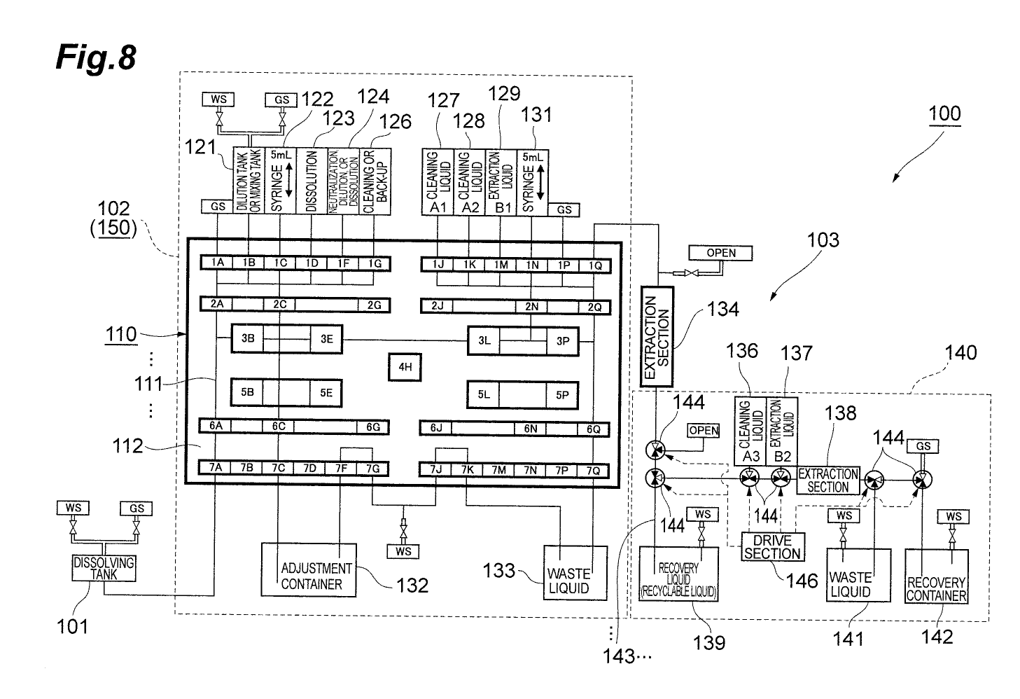

FIG. 8 is a schematic configuration view showing a system configuration of a radioactive isotope refining system 100 including a solution adjusting apparatus 150 related to the present embodiment. The radioactive isotope refining system 100 is a system that can refine a plurality of different types of radioactive isotopes. As shown in FIG. 8, the radioactive isotope refining system 100 includes a dissolving tank 101 that dissolves a target material in which radioactive isotopes are generated as a charged particle beam is radiated, a solution adjustment unit 102 that performs adjustment of a solution in which the radioactive isotope is dissolved, and a refinement section 103 that refines the radioactive isotope contained in the solution adjusted in the solution adjustment unit 102. In addition, in FIG. 8, the portion of the piping shown by one solid line is replaceable piping, and the portion shown by two solid lines is piping that is fixed to the system and is not premised on replacement in a short-term cycle. In addition, the “replaceable” in the present specification means being premised on “disposable” of being replaceable with a new one if being used one time or a prescribed number of times.

Examples of the radioactive isotope that can be refined in the radioactive isotope refining system 100 including the solution adjusting apparatus 150 related to the present embodiment include ⁶⁴Cu, ⁸⁹Zr, ^{99m}Tc, and the like. In addition, charged particles to be irradiated are protons, deutons, alpha particles, 3He, electrons, or the like.

When radioactive isotopes are generated, a metal layer is formed as a target material on the surface of a target substrate constituted by a metal plate. The metal layer formed by the target material is formed on the surface of the target substrate by performing plating processing. By irradiating the target material with the charged particle beam, a small amount of radioactive isotopes are generated in the target material. In addition, examples of the material of the target substrate include Au, Al, Pt, and the like. Examples of the target material include ⁶⁴Ni, ⁸⁹Y, ¹⁰⁰Mo, and the like. In the dissolving tank 101, the target material is dissolved together with radioactive isotopes using a dissolving liquid. Thereby, a solution in which the radioactive isotopes and the target material are mixed is obtained. Examples of the dissolving liquid include hydrochloric acid, nitric acid, sodium hydroxide, hydrogen peroxide, sulfuric acid, and the like.

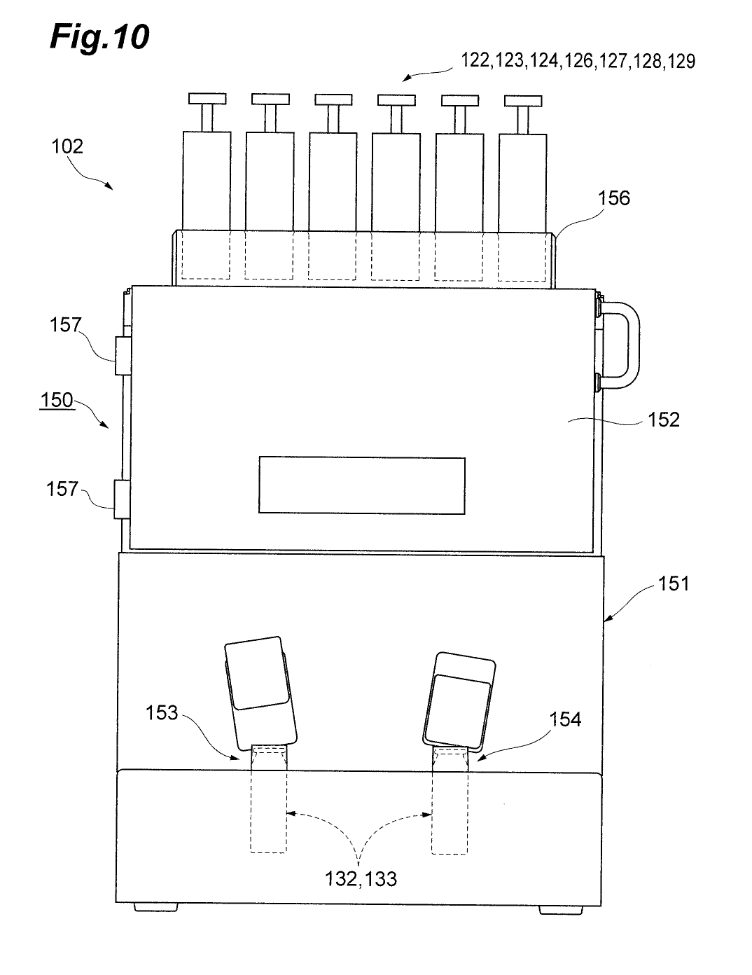

The solution adjustment unit 102 is configured by assembling various containers, piping, or the like to the solution adjusting apparatus 150 (refer to FIG. 10) including sensors, pumps, a drive section, valves, mechanisms, or the like required in order to perform concentration adjustment or the like of the solution generated in the dissolving tank 101. The solution adjustment unit 102 is capable of detachably fixing a cassette 110 for a radioactive isotope refining system (cassette for a radioactive isotope handling apparatus) in that a plurality of piece of piping 111 are provided (the detailed configuration thereof will be described below). In the following description, the “cassette for a radioactive isotope refining system” is referred to as a “cassette”. Additionally, various containers to be used for adjustment of the solution are assembled to the solution adjustment unit 102. In an example shown in FIG. 8, the solution adjustment unit 102 includes a container 121 used as a dilution tank or a mixing tank, a syringe 122, a container 123 in which a dissolving liquid is contained, a container 124 in which a liquid for neutralization, dilution, or dissolution is contained, a container 126 in which a cleaning liquid is contained (or used as a back-up), a the container 127 in which a cleaning liquid A1 is contained, a container 127 in which a cleaning liquid A2 is contained, a container 129 in which an extraction liquid B1 is contained, a syringe 131, an adjustment container 132 that adjusts the solution by dilution, mixture, or the like, and a waste liquid container 133 that recovers a waste liquid. In addition, since the flow channels of the cassette 110 can be freely changed in the present embodiment, the liquids contained in containers 22 to 29 may be replaced with each other.

As the liquids that are contained in the containers 123, 124, and 126 and are used to adjust the solution, the same dissolving liquids as the above-described dissolving liquids may be used (oxygenated water may be added to them), or water or the like may be used. As the cleaning liquids A1 and A2 contained in the containers 127 and 128, the same dissolving liquids as the above-mentioned dissolving liquids may be used, or a physiological salt solution, water, or the like may be used. As the extraction liquid B1 contained in the container 129, hydrochloric acid, nitric acid, oxalic acid, dichloromethane (CH₂Cl₂) containing tetrabutylammonium bromide (TBAB), chloroform, or the like may be used.

The refinement section 103 includes a first adjustment container 132 that extracts radioactive isotopes from the solution, a container 136 that contains a cleaning liquid A3, a container 137 that contains an extraction liquid B2, a second extraction section 138 that extracts radioactive isotopes from the solution, a recovery container 139 that recovers a recyclable recovery liquid, a waste liquid container 141 that recovers a waste liquid, a recovery container 142 that recovers the solution of the refined radioactive isotopes, piping 143 that connects the respective constituent elements, a three-way stopcock 144 that is provided in the piping 143, and a drive section 146 that applies a driving force for switching the three-way stopcock 144. In addition, some or all of the constituent elements of the refinement section 103 may be unitized. In the example shown in FIG. 8, a refinement unit 140 is provided so as to be configured by assembling various containers and extraction sections to a refining apparatus including sensors, pumps, the drive section 146, valves, and mechanisms required for refinement of radioactive isotopes. The refinement unit 140 includes the containers 136, 137, 139, and 141, 142, the second extraction section 138, the piping 143, the three-way stopcock 144, and the drive section 146.

As the first adjustment container 132, anionic exchange resin, or selective resin of radioactive isotopes can be applied. The second adjustment container 132 is arranged downstream of the first adjustment container 132, and further refines the radioactive isotopes. As the second extraction section 138, Sep-Pak (registered trademark) or the like can be applied.

The containers 136 and 137 are arranged downstream of the first adjustment container 132 and is arranged upstream of the second extraction section 138. As the cleaning liquid A3 contained in the container 136, a physiological salt solution, water, or the like may be used. As the extraction liquid 32 contained in the container 137, hydrochloric acid, a physiological salt solution, or the like may be used.

The three-way stopcock 144 is provided in a flow channel downstream of the first adjustment container 132, and switches the flow of the flow channel. The three-way stopcock 144 connects two pieces of piping 143 provided downstream of the first adjustment container 132, and switches valves therein, thereby allowing two pieces of piping 143 of three pieces of piping 143 to communicate with each other. The three-way stopcock 144 and the piping 143 are replaceable disposable constituent elements. The drive section 146 is provided separately from the three-way stopcock 144, and applies a driving force for switching of the three-way stopcock. The drive section 146 is provided in the refinement unit 140 as a common part, irrespective of the type of radioactive isotopes to be refined, unlike the disposable three-way stopcock 144. The three-way stopcock 144 is attached to an attaching portion of the drive section 146 when used, and the used three-way stopcock 144 is removed from the attaching portion when replaced. For example, the three-way stopcock 144 has the structure in which the valves are switched depending on the pressure of air. The drive section 146 has the supply structure in which air is supplied to three-way stopcock 144. For example, a main body portion of the supply structure may be stored inside a housing of the refining apparatus, and an attaching portion that attaches the three-way stopcock 144 may be provided on an outer wall portion of the housing so as to be exposed.

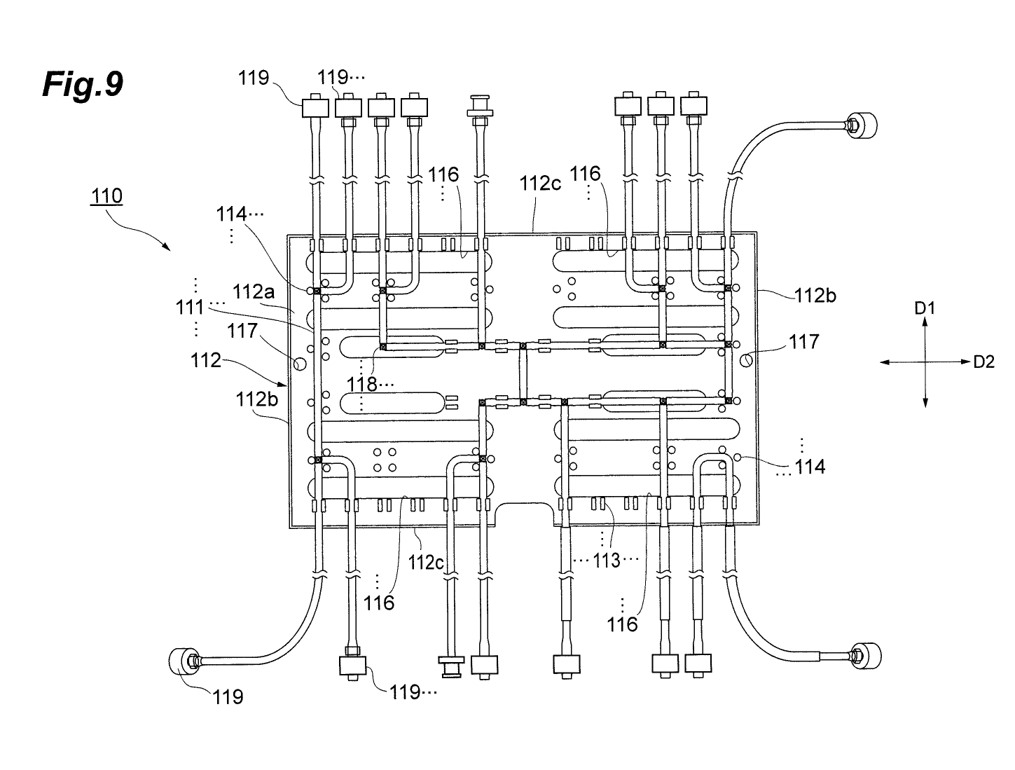

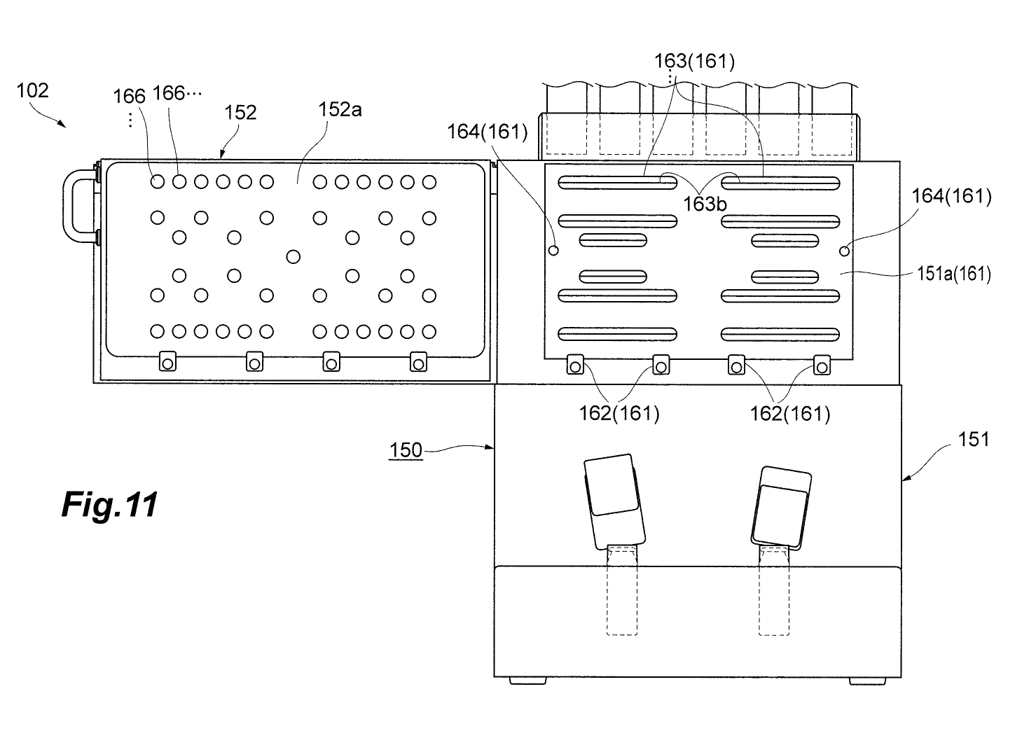

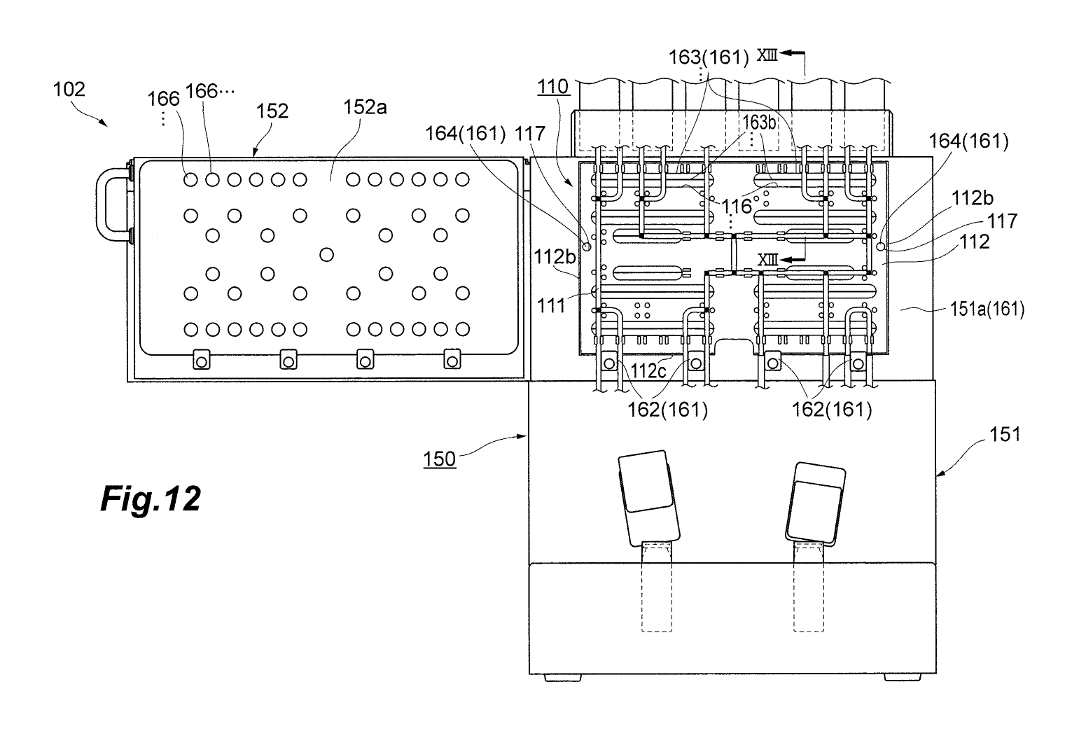

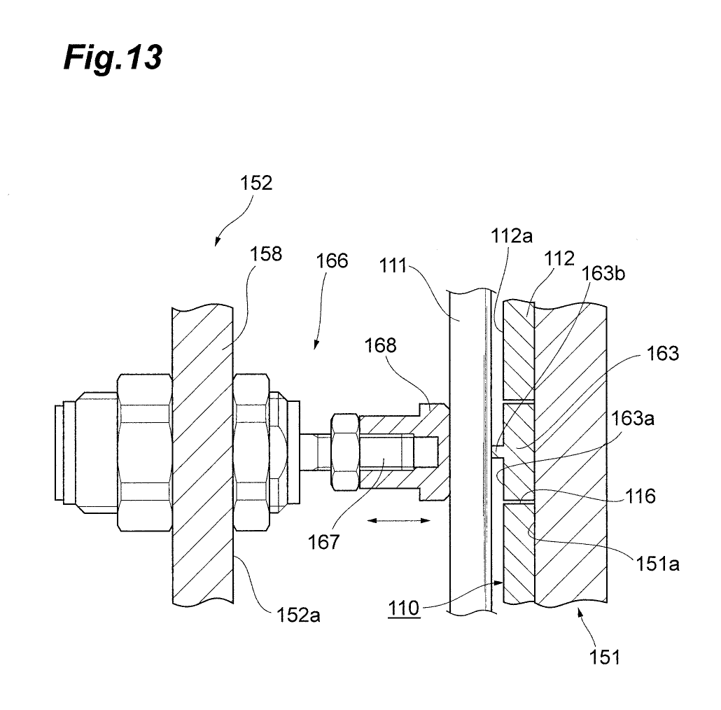

Here, the detailed configuration of the solution adjustment unit 102 and the cassette 110 will be described with reference to FIGS. 9 to 13. FIG. 9 is a plan view showing an example of the configuration of the cassette 110 related to the present embodiment. FIG. 10 is a front view showing an example of the configuration of the solution adjustment unit 102 including the solution adjusting apparatus 150 related to the present embodiment. FIG. 11 is a front view showing a state where a door portion 152 of the solution adjustment unit 102 shown in FIG. 10 is opened. FIG. 12 is a front view showing an aspect in which the cassette 110 is fixed to a fixing portion 161 of the solution adjustment unit 102 shown in FIG. 11. FIG. 13 is a cross-sectional view taken along line XIII-XIII shown in FIG. 12, and is a cross-sectional view in a state where the door portion 152 is closed.

The cassette 110 is a disposable module that is replaceably applied to the radioactive isotope refining system 100 and is replaceably attached to the solution adjusting apparatus 150 of the solution adjustment unit 102. As shown in FIG. 9, the cassette 110 includes a substrate 112 and a plurality of pieces of piping 111 attached to the substrate 112.

Since the substrate 112 is a rectangle plate-shaped member and is a disposable member, for example, polypropylene or the like is applied as the material of the substrate. In an example shown in FIG. 9, the substrate 112 has a rectangular shape that has a pair of long sides that extend in parallel to each other along a second direction D2, and a pair of short sides that extend in parallel to each other along a first direction D1 orthogonal to the second direction D2. Hooks (holding means) 113 for pinching and guiding the piping 111, and strut portions (holding means) 114 are formed on a surface 112a of the substrate 112. Each hook 113 is configured so that two claws face each other and protrude from the surface 112a, and the piping 111 can be fixed by pinching the piping 111 between the claws. Additionally, the hooks 113 function as first holding means capable of attaching the piping 111 along the first direction D1, and functions as second holding means capable of attaching the piping 111 along the second direction D2. Each strut portion 114 is configured so that a plurality of struts face and protrude, and the piping 111 can be fixed by pinching the piping 111 between struts. The strut portions 114 can function as the first holding means and second holding means simultaneously capable of attaching the piping 111 along the first direction D1 and capable of attaching the piping 111 along the second direction D2, depending on the attaching method of the piping 111. The hooks 113 and the strut portions 114 are provided at predetermined positions of the surface 112a of the substrate 112, and depending on the routing of the piping 111, some of the hooks and the strut portions may be used and some thereof may not be used.

The substrate 112 is formed with elongated holes 116 that penetrate in the thickness direction of the substrate 112. In the present embodiment, the elongated holes 116 extend along the first direction D1, and are formed at predetermined intervals in the second direction D2. Additionally, circular through holes 117 for positioning are formed in edges 112b corresponding to the short sides of the substrate 112. In addition, although the hooks 113, the strut portions 114, the elongated holes 116, and the through holes 117 are provided symmetrically with respect to the centerline in the first direction D1, these are not limited to such arrangement.

The piping 111 is fixed in the state of being positioned on the substrate 112 as being pinched by the hooks 113 or the strut portions 114. Since the piping 111 is a disposable member, for example, a silicone tube, a Teflon (registered trademark), Tefzel, polyurethane, or the like is applied. Additionally, by connecting the piping 111 to each other via joints 118, flow channels that are curved at a right angle or flow channels that branch in three ways can be formed on the substrate 112. By changing fixing positions by the hooks 113 and the strut portions 114 or connecting positions by the joints 118, it is possible to freely set a piping configuration with respect to the substrate 112. Additionally, the piping 111 passes above the elongated holes 116. Some pieces of piping 111 extend to the outside from edges 112c corresponding to the long sides of the substrate 112, and connectors 119 are provided at the tip of the piping 111. The connectors 119 are detachably connected to the dissolving tank 101, the various containers, the first adjustment container 132, and the like, which are shown in FIG. 8. In addition, the configuration of the cassette 110 shown in FIG. 9 is merely an example, the shape of a substrate 112 is not limited to one shown in the drawing, and the positions of the hooks 113, the strut portions 114, or the like are not limited to those shown in the drawing. Additionally, the piping configuration by the combination of the plurality of pieces of piping 111 is also not limited to one shown in the drawing.

As shown in FIGS. 10 to 12, the solution adjustment unit 102 is configured by assembling the various containers, piping, and the like, which are shown in FIG. 8, to the solution adjusting apparatus 150. Additionally, the solution adjustment unit 102 is capable of detachably attaching the cassette 110. The cassette 110 is a replaceable module, whereas the solution adjusting apparatus 150 is configured as a fixed module that is used in common, irrespective of the type of radioactive isotopes to be refined. The various containers or piping other than the cassette 110 may be replaced depending on the type of radioactive isotopes, or the same containers or piping may be cleaned and used. The solution adjusting apparatus 150 has a main body portion 151 and the door portion 152. The door portion 152 is provided so as to cover an upper region of the main body portion 151.

Accommodating portions 153 and 154 that accommodate the containers 132 and 133 are provided in a lower region of the main body portion 151. A cooler for cooling the adjustment container 132, a heater for heating the adjustment container 132, a pressure sensor for checking the pressure within the adjustment container 132, a thermometer for checking the temperature within the adjustment container 132, a radiation sensor for checking the dose of radiation contained within the adjustment container 132, and the like are provided around the accommodating portion 153. Additionally, the main body portion 151 is provided with an attaching portion 156 that attaches the various containers 22 to 29. Whether any container is provided in any portion of the attaching portion 156 is not particularly limited.

The door portion 152 is provided so as to be openable and closable via hinges 157 (refer to FIG. 10) provided at a side surface of the main body portion 151. As shown in FIGS. 11 and 12, a fixing portion 161 capable of detachably fixing the cassette 110 is provided on a front surface 151a of the main body portion 151 at a position covered with the door portion 152. The fixing portion 161 includes the front surface 151a that receives the substrate 112 of the cassette 110, claw portions 162 that support the edges 112c of the substrate 112 of the cassette 110, piping elliptical receiving portions (protruding portions) 163 that are inserted through the elongated holes 116 of the substrate 112, and the columnar pins 164 that are inserted through the through holes 117 of the substrate 112.

Each claw portion 162 has an L-shape as viewed from the side surface of the main body portion 151, and has a portion that protrudes forward from the front surface 151a and a portion that extends upward from the tip of the protruding portion. The substrate 112 is fixed by being pinched by the portion that extends upward, and the front surface 151a. A plurality of piping receiving portions 163 are provided so as to protrude forward from the front surface 151a, and are provided in shapes and at positions (the positions of the elongated holes 116 when the substrate 112 are fixed by the claw portions 162) corresponding to the elongated holes 116 of the substrate 112. A plurality of (two in the present embodiment) pins 164 are provided so as to protrude forward the front surface 151a, and are provided in shapes and at positions (the positions of the through holes 117 when the substrate 112 is fixed by the claw portions 162) corresponding the through holes 117 of the substrate 112. As the pins 164 are inserted through the through holes 117, the cassette 110 is positioned with respect to the fixing portion 161.

An inner surface 152a of the door portion 152 faces the front surface 151a of the main body portion 151 so as to become parallel to the front surface at a predetermined interval when the door portion 152 is closed. A plurality of pressing members 166 are provided at predetermined positions of the inner surface 152a of the door portion 152. The plurality pressing members 166 are, for example, air cylinders that move back and forth with the force of air. Air is supplied to the individual pressing members 166 via an air tube (not shown) extends from the main body portion 151. In addition, the driving of the pressing members 166 may not be performed by the force of air, and may be performed by an electrical force or a magnetic force. The pressing members 166 are provided at positions that face the piping receiving portions 163 of the main body portion 151, in a state where the door portion 152 is closed. Additionally, the piping 111 of the cassette 110 is arranged between the pressing members 166 and the piping receiving portions 163. Accordingly, the pressing members 166 is capable of pressing the piping 111 between the pressing members and the piping receiving portions 163 of the main body portion 151 in a state where the door portion 152 is closed.

The pressing structure formed by the pressing members 166 will be described in more detail with reference to FIG. 13. As shown in FIG. 13, each pressing member 166 includes a shank 167 that is supported by a wall portion 158 (having the inner surface 152a of the door portion 152) of the door portion 152 and protrudes from the inner surface 152a, and a pressing portion 168 provided at the shank 167. The pressing member 166 is capable of reciprocating forward and backward along the extending direction of the shank 167. On the other hand, the piping receiving portion 163 is provided at a position that faces the pressing portion 168 of the pressing members 166, on the front surface 151a of the main body portion 151. A protruding portion 163b is formed on an end face 163a of the piping receiving portion 163. The tip face of the protruding portion 163b preferably protrude further toward the pressing member 166 side than the surface 112a of the substrate 112 in the state of being fixed to the fixing portion 161. The center position of the pressing member 166 substantially coincides with the center position of the protruding portion 163b in the height direction. Accordingly, when the pressing portions 168 of the pressing members 166 move to the piping receiving portions 163 side, the piping 111 is pinched and pressed between the pressing portions 168 and the piping receiving portions 163, and the flow channels are closed as the internal space of the piping 111 is crushed. In addition, the pressing structure formed by the pressing members 166 is not particularly limited, and the piping receiving portions 163 may not have the protruding portions 163b. Additionally, the piping receiving portions 163 and the elongated holes 116 themselves may not be provided, and the piping 111 may be pinched between the pressing portions 168 of the pressing members 166, and the surface 112a of the substrate 112.

As described above, the solution adjustment unit 102 is capable of freely setting the flow channels configured by the piping 111 of the cassette 110 by pressing the piping 111 with the pressing members 166. In FIG. 8, the pressing positions where the piping 111 is capable of pressed by the pressing members 166 are shown by 1A to 1Q, 2A to 2Q, 3B to 3P, 4H, 5B to 5P, 6A to 6Q, and 7A to 7Q. In a case where a line through which a solution is expected to pass is determined, the pressing by the pressing members 166 is set to OFF at the pressing positions that are present on the line, and the pressing by the pressing members 166 is set to ON at the other pressing positions. Thereby, the solution flows through the flow channel along a desired line, and does not flow to any flow channels other than the line.

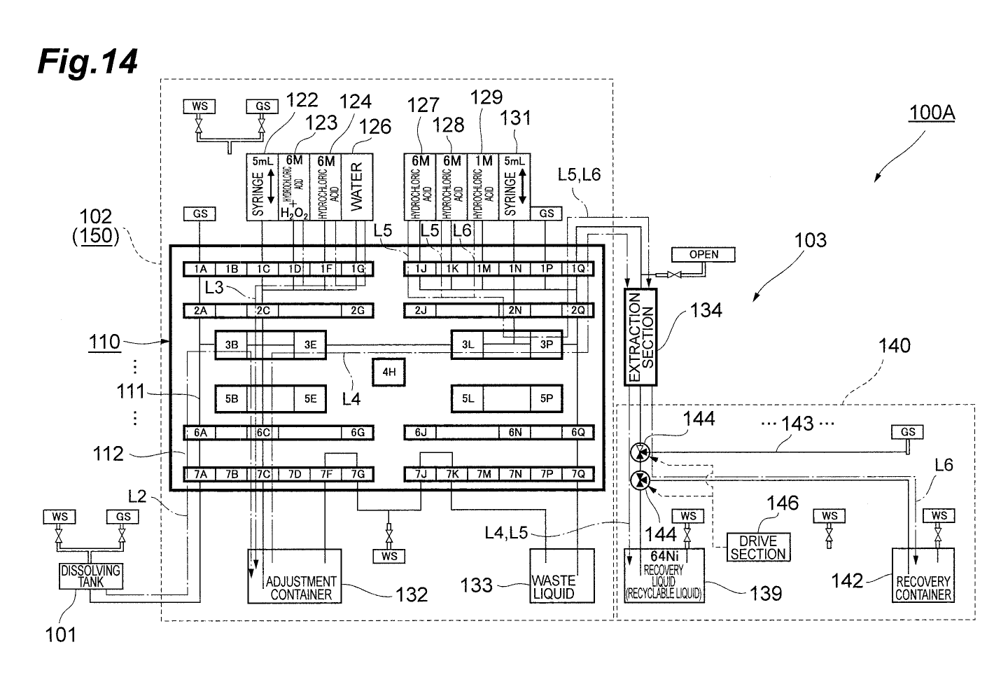

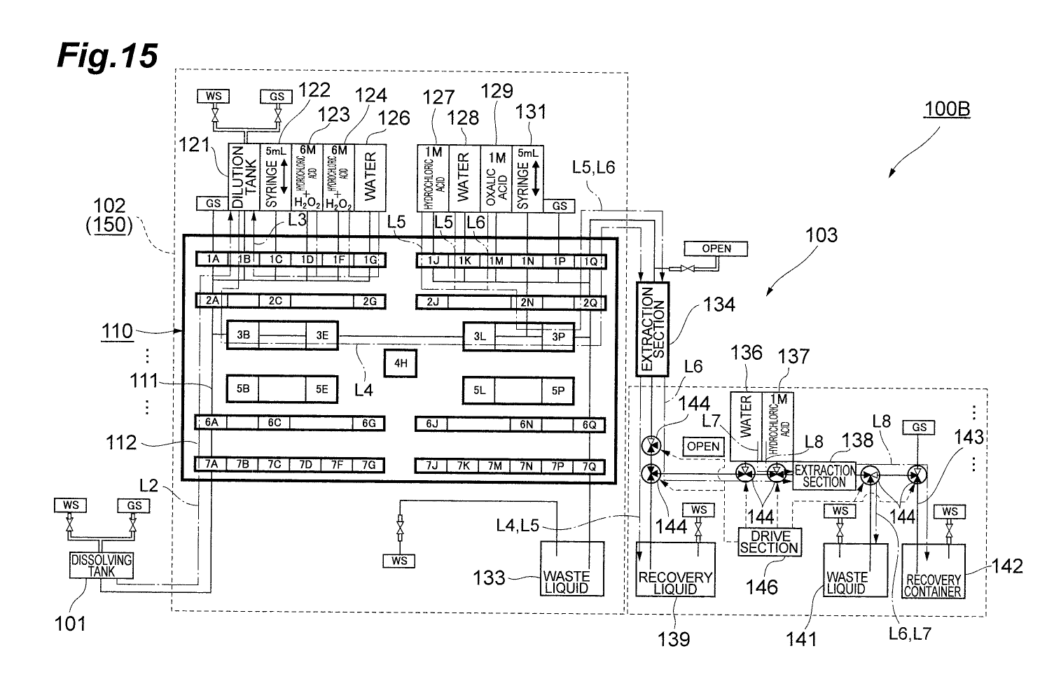

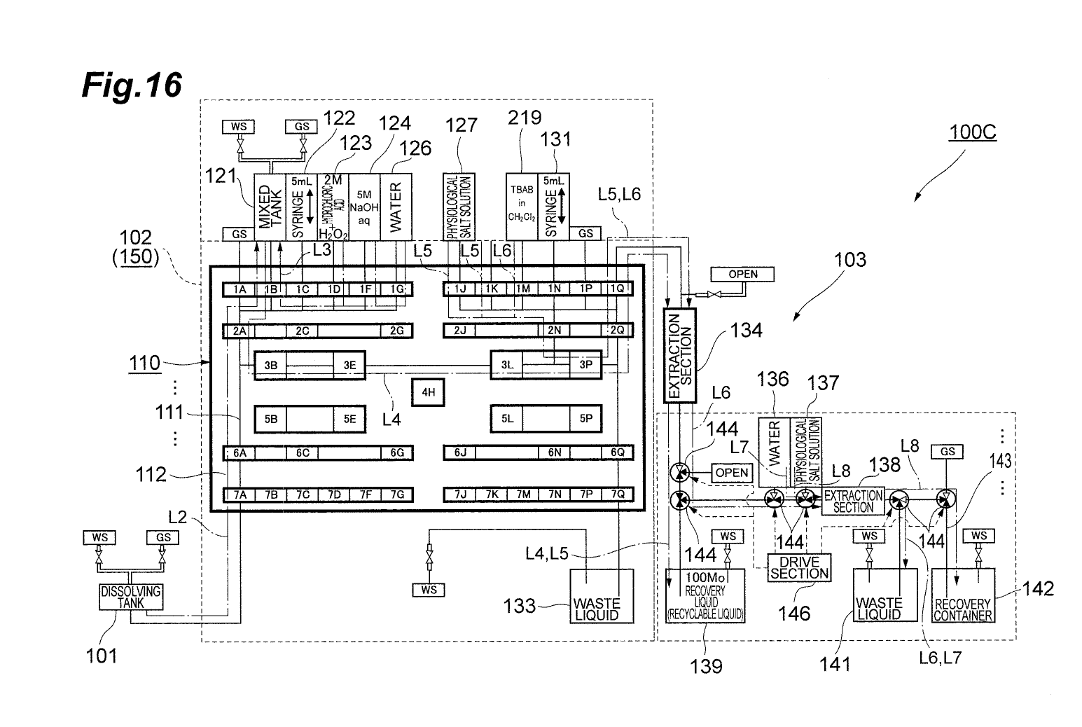

Next, an example of a procedure in a case where radioactive isotopes are refined using the radioactive isotope refining system 100 shown in FIG. 8 will be described. FIG. 14 shows an example of the schematic configuration of a radioactive isotope refining system 100A in a case where ⁶⁴Cu is refined, FIG. 15 shows an example of the schematic configuration of a radioactive isotope refining system 100B in a case where ⁸⁹Zr is refined, and FIG. 16 shows an example of the schematic configuration of a radioactive isotope refining system 100C in a case where ^{99m}Tc is refined.

Refinement of ⁶⁴Cu

The radioactive isotope refining system 100A as shown in FIG. 14 is first configured by assembling various piping and containers. The container 123 that contains 6 mol/L of hydrochloric acid (including oxygenated water), a container 124 that contains 6 mol/L of hydrochloric acid, the container 126 that contains water, the containers 127 and 128 that contains 6 mol/L of hydrochloric acid, and the container 129 that contains 1 mol/L of hydrochloric acid are assembled to the solution adjustment unit 102. Additionally, the adjustment container 132 and the waste liquid container 133 are assembled to the solution adjustment unit 102. Additionally, the cassette 110 is attached to the solution adjustment unit 102, and the connectors 119 (refer to FIG. 9) provided at the respective pieces of piping 111 are connected to the mating connectors. In the refinement section 103, anionic exchange resin is prepared as the first adjustment container 132, the recovery container 139 and the recovery container 142 are assembled to the refinement unit 140, and the piping 143 and the three-way stopcock 144 are assembled to the refinement unit 140 in a predetermined pattern. In addition, in the radioactive isotope refining system 100A, the second extraction section 138 is not assembled, and thus the containers 136 and 137 are not assembled.

Procedure 1

First, a solution in which Ni and ⁶⁴Cu are mixed is obtained by dissolving a metal layer of Ni(⁶⁴Ni), which is formed on a target substrate surface of Au and is irradiated with a charged particle beam, with 6 mol/L of hydrochloric acid in the dissolving tank 101 while heating the metal layer.

Procedure 2

The solution adjustment unit 102 makes the solution obtained in dissolving tank 101 flow to the adjustment container 132 via the line L2 shown in the drawing. At this time, the solution adjustment unit 102 controls ON/OFF of pressing at respective pressing positions so that a flow channel related to the line L2 is set. Specifically, pressing at the pressing positions 7A, 6A, 3B, 6C, and 7C through which the line L2 passes is set to OFF, and pressing at the other pressing positions is set to ON. In addition, since control methods when flow channels are set in subsequent procedures have the same purport as that of the line L2, the description thereof is omitted.

Procedure 3

The solution adjustment unit 102 makes the liquids of the containers 123, 124, and 126 flow to the adjustment container 132, to thereby perform concentration adjustment of the solution within the adjustment container 132. The solution adjustment unit 102 sets a flow channel related to the line L3 shown in the drawing, and makes 6 mol/L of hydrochloric acid (the pressing position 10: OFF and the pressing positions 1F and 1G: ON) including oxygenated water, of the container 123, 6 mol/L of hydrochloric acid (the pressing position 1F: OFF and the pressing positions 1D and 1G: ON) of the container 124 and the water (the pressing position 1G: OFF and the pressing positions 1D and 1 F: ON) of the container 126 flow in this order to the first adjustment container 132 via the line L3. In addition, in a case where each of the containers 123, 124, 126 is constituted by a syringe, a liquid can be made to flow directly from each of the containers 123, 124, and 126 to the adjustment container 132. In a case where each of the containers 123, 124, 126 is merely a container, a liquid is made to flow to the adjustment container 132 after a desired amount of liquid is sucked off by the syringe 122. In addition, the syringe 131 is used even in case where each of the containers 127, 128, and 129 is merely a container.

Procedure 4

The solution adjustment unit 102 makes the solution adjusted in the adjustment container 132 flow to the first adjustment container 132. The solution adjustment unit 102 sets a flow channel related to the line L4 shown in the drawing, and makes a solution in which Ni and ⁶⁴Cu are mixed to the first adjustment container 132 via the line L4. ⁶⁴CU is present as a tetrachloro copper ion ([CuCl₄]²⁻) that is a negative ion, and Ni is present as a nickel ion ([Ni²⁺]) that is a positive ion. ⁶⁴CU is adsorbed on the ion exchange resin, and Ni is not adsorbed but passes through the first adjustment container 132 together with the hydrochloric acid solution, and is recovered in the recovery container 139. However, some Ni remains within the first adjustment container 132.

Procedure 5

The solution adjustment unit 102 makes the liquids of the containers 127 and 128 flow to the first adjustment container 132, to thereby elute Ni that remains in the first adjustment container 132. The solution adjustment unit 102 sets a flow channel related to the line L5 shown in the drawing, and makes 6 mol/L of hydrochloric acid (the pressing position 1J: OFF and the pressing position 1K: ON) of the container 127 and 6 mol/L of hydrochloric acid (the pressing position 1K: OFF and the pressing position 1J: ON) of the container 127 flow in this order to the adjustment container 132 via the line L5. The hydrochloric acid solution that has passed through the first adjustment container 132 is recovered in the recovery container 139 together with Ni.

Procedure 6

The solution adjustment unit 102 makes the liquid of the container 129 flow to the first adjustment container 132, to thereby elute ⁶⁴Cu adsorbed on the first adjustment container 132. The solution adjustment unit 102 sets a flow channel related to the line L6 shown in the drawing, and makes 1 mol/L of hydrochloric acid of the container 129 flow to the first adjustment container 132 via the line L6. The hydrochloric acid solution that has passed through the first adjustment container 132 is recovered in the recovery container 142 together with ⁶⁴Cu. In addition, the three-way stopcock 144 switches a flow channel from the line L5 that turns to the recovery container 139 to the line L6 that turns to the recovery container 142 by a driving force applied from the drive section 146 between Procedure 5 and Procedure 6. Refined ⁶⁴Cu is obtained by the above.

Refinement of ⁸⁹Zr

The radioactive isotope refining system 100B as shown in FIG. 15 is first configured by assembling various piping and containers. The container 121 used as the dilution tank, the container 123 that contains 6 mol/L of hydrochloric acid (including oxygenated water), the container 124 that contains 6 mol/L of hydrochloric acid (including oxygenated water), the container 126 that contains water, the container 127 that contains 1 mol/L of hydrochloric acid, the container 128 that contains water, and the container 129 that contains 1 mol/L of oxalic acid are assembled to the solution adjustment unit 102. Additionally, the waste liquid container 133 is assembled to the solution adjustment unit 102. In addition, in the radioactive isotope refining system 100B, the container 121 as the dilution tank is used instead of the adjustment container 132. Additionally, the cassette 110 is attached to the solution adjustment unit 102, and the connectors 119 (refer to FIG. 9) provided at the respective pieces of piping 111 are connected to the mating connectors. In the refinement section 103, the container 136 that prepares Zr selecting-and-holding resin as the first adjustment container 132, and contains water, the container 137 that contains 1 mol/L of hydrochloric acid, the second extraction section 138 to which Sep-Pak (registered trademark) QMA is applied, the recovery container 139, the waste liquid container 141, and the recovery container 142 are assembled to the refinement unit 140, and the piping 143 and the three-way stopcock 144 are assembled to the refinement unit 140 in a predetermined pattern.

Procedure 1

First, a solution in which Y and ⁸⁹Zr are mixed is obtained by dissolving a metal layer of Y (⁶⁹Y), which is formed on a target substrate surface and is irradiated with a charged particle beam, with 6 mol/L of hydrochloric acid (including oxygenated water) in the dissolving tank 101.

Procedures 2 to 5

The processing of the same purport as that of the radioactive isotope refining system 100A that refines ⁶⁴Cu is performed except that the adjustment of the solution is performed by the container 121 as the dilution tank instead of the adjustment container 132.

Procedure 6

The solution adjustment unit 102 makes the liquid of the container 129 flow to the first adjustment container 132, to thereby elute ⁸⁹Zr held by the first adjustment container 132. The solution adjustment unit 102 sets a flow channel related to the line L6 shown in the drawing, and makes 1 mol/L of oxalic acid of the container 129 flow to the first adjustment container 132 via the line L6. The oxalic acid solution that has passed through the first adjustment container 132 passes through the second extraction section 138 together with ⁸⁹Zr. ⁸⁹Zr and remaining impurities remain in the second extraction section 138. The oxalic acid solution and some impurities are recovered in the waste liquid container 141. In addition, the three-way stopcock 144 switches a flow channel from the line L5 that turns to the recovery container 139 to the line L6 that turns to the second extraction section 138 and the waste liquid container 141 by a driving force applied from the drive section 146 between Procedure 5 and Procedure 6.

Procedure 7

The refinement unit 140 makes the liquid of the container 136 flow to the second extraction section 138, to thereby make the remaining impurities in the second extraction section 138 flow. The refinement unit 140 switches the three-way stopcock 144 by the drive section 146, to thereby set a flow channel related to the line L7 shown in the drawing and make the water of the container 136 flow to the second extraction section 138 via the line L7. The water that has passed through the second extraction section 138 is recovered in the waste liquid container 141 together with the impurities.

Procedure 8

The refinement unit 140 makes the liquid of the container 137 flow to the second extraction section 138, to thereby elute ⁸⁹Zr held by the second adjustment container 132. The refinement unit 140 sets a flow channel related to the line L8 shown in the drawing, and makes 1 mol/L of hydrochloric acid of the container 137 flow to the second extraction section 138 via the line L8. The hydrochloric acid solution that has passed through the second extraction section 138 is recovered in the recovery container 142 together with ⁸⁹Zr. Refined ⁶⁹Zr is obtained by the above.

Refinement of ^{99m}Tc

The radioactive isotope refining system 100C as shown in FIG. 16 is first configured by assembling various piping and containers. The container 121 used as the mixing tank, and the container 123 that contains 2 mol/L of hydrochloric acid (including oxygenated water), the container 124 that contains 5 mol/L of sodium hydroxide (including oxygenated water), the container 126 that contains water, the container 127 that contains the physiological salt solution, and the container 129 that contains the dichloromethane (CH₂C₁₂) containing tetrabutylammonium bromide (TBAB) are assembled to the solution adjustment unit 102. Additionally, the waste liquid container 133 is assembled to the solution adjustment unit 102. In addition, in the radioactive isotope refining system 100C, the container 121 as the mixing tank is used instead of the adjustment container 132. Additionally, the cassette 110 is attached to the solution adjustment unit 102, and the connectors 119 (refer to FIG. 9) provided at the respective pieces of piping 111 are connected to the mating connectors. In the refinement section 103, the container 136 that prepares anionic exchange resin as the first adjustment container 132, and contains water, the container 137 that contains the physiological salt solution, the second extraction section 138 to which Sep-Pak (registered trademark) Al—N is applied, the recovery container 139, the waste liquid container 141, and the recovery container 142 are assembled to the refinement unit 140, and the piping 143 and the three-way stopcock 144 are assembled to the refinement unit 140 in a predetermined pattern.

Procedure 1

First, a solution in which Mo and ^{99m}Tc are mixed is obtained by dissolving a metal layer of Mo (¹⁰⁰Mo), which is formed on a target substrate surface and is irradiated with a charged particle beam, with a predetermined concentration of hydrochloric acid (including oxygenated water) in the dissolving tank 101.

Procedures 2 to 8

Since the processing of the same purport as that of the radioactive isotope refining system 100B that refines ⁸⁹Zr, the description thereof is omitted.

Next, the operation and effects of the radioactive isotope refining system 100 including the solution adjusting apparatus 150 related to the present embodiment and the cassette 110 for the radioactive isotope refining system will be described.

In a case where different types of radioactive isotopes are refined, refining procedures may be different for the respective types. Accordingly, in the radioactive isotope refining system of the related art, there is a case where individual systems individual solution adjustment units corresponding to the respective types are required. Alternatively, in the radioactive isotope refining system of the related art, the necessity for cleaning piping or the like occurs in a case where different types of radioactive isotopes are intended to be refined by one solution adjustment unit. Moreover, in a case where the cleaning is insufficient, there is a case where liquids used for refining other types of the radioactive isotopes types remain and refining performance degrades.

On the other hand, according to the radioactive isotope refining system 100 including the solution adjusting apparatus 150 related to the present embodiment, the solution adjustment unit 102 is capable of pressing the piping 111 of the cassette 110 fixed by the fixing portion 161, using the pressing members 166, to thereby freely set flow channels. Accordingly, in a case where a plurality of different types of radioactive isotopes are refined, the solution adjustment unit 102 can set flow channels so that suitable refining procedures can be executed according to the types. This enables a plurality of types of radioactive isotopes to be refined using one solution adjustment unit 102. Additionally, since the plurality of types of radioactive isotopes can be refined using one solution adjustment unit 102 in this way, it is possible to reduce the size of the overall system.

Additionally, the solution adjustment unit 102 has the fixing portion 161 capable of detachably fixing the cassette 110. By virtue of such a configuration, a disposable cassette 110 that is replaceable with respect to the solution adjustment unit 102 can be adopted as the cassette 110. Accordingly, since the disposable cassette can be replaced with a new cassette 110 in a case where a radioactive isotope is refined, cleaning of the piping or the like can be made unnecessary and degradation of the refining performance caused by the influence of residue can also be prevented. From the above, different types of radioactive isotopes can be refined without degrading refining performance.

In the radioactive isotope refining system 100 including the solution adjusting apparatus 150 related to the present embodiment, the solution adjustment unit 102 includes the piping receiving portions 163 at positions corresponding to the elongated holes 116 formed in the cassette 110, and the pressing members 166 are provided at positions corresponding to the piping receiving portions 163. By virtue of such a configuration, the pressing members 166 can pinch the piping 111 of the cassette 110 between the pressing members and the piping receiving portions 163. This enables the pressing members 166 to reliably block the piping 111, and enables the flow channels to be reliably set.

The radioactive isotope refining system 100 including the solution adjusting apparatus 150 related to the present embodiment further includes the refinement section 103 that refines the radioactive isotopes contained in the solution adjusted in the solution adjustment unit 102. Additionally, the refinement section 103 includes the first adjustment container 132 that extracts radioactive isotopes from the solution, the replaceable three-way stopcock 144 that is provided a flow channel downstream of the first adjustment container 132, and the drive section 146 that is provided separately from the three-way stopcock 144 and applies a driving force for the switching of the three-way stopcock 144. In a case where a portion that switches the direction of flow is present in the flow channel downstream of the first adjustment container 132, an inexpensive disposable three-way stopcock 144 is used for a portion through which a fluid passes, and the drive section 146 that is separate from the three-way stopcock 144 is used for a portion that applies a driving force to the three-way stopcock 144. Thereby, in a case where different types of radioactive isotopes are refined, the drive section 146 can be used as a common part irrespective of the type of radioactive isotopes, and the portion through which a liquid passes can be replaced with a new three-way stopcock 144. This can prevent degradation of refining performance with inexpensive structure.

In the solution adjusting apparatus 150 related to the present embodiment, the fixing portion 161 has the front surface 151a that receives the substrate 112 of the cassette 110, the claw portions 162 that supports the edges 112c of the substrate 112, and the plurality of piping receiving portions 163 that protrude from the front surface 151a. The fixing portion 161 can receive the substrate 112 in the front surface 151a, can support the edges 112c of the substrate 112 with the claw portions 162, and can make the piping receiving portions 163 received through the elongated holes 116 of the substrate 112. This enables the fixing portion 161 to fix the cassette 110 reliably.

The solution adjusting apparatus 150 related to the present embodiment further includes the main body portion 151 to which the cassette 110 is attached, and the door portion 152 that are openably and closably provided at the main body portion 151. Additionally, the plurality of pressing members 166 are provided at the door portion 152, and are capable of pressing the piping 111 in a state where the door portion 152 is closed. In this case, the positions of the pressing members 166 can be changed in a state where the door portion 152 is opened and a state where the door portion 152 is closed. That is, in a case where the cassette 110 is attached and the solution adjusting apparatus 150 is operated and a case where the cassette 110 is removed, the positions of the pressing members 166 can be changed, and it is possible to improve the workability of removal of the cassette 110.

In the solution adjusting apparatus 150, the main body portion 151 may include the piping receiving portions 163 at positions corresponding to the elongated holes 116 provided in the cassette 110, and the pressing members 166 may be provided at positions corresponding to the piping receiving portions 163.

By virtue of such a configuration, the pressing members 166 can pinch the piping 111 of the cassette 110 between the pressing members and the piping receiving portions 163. This enables the pressing members 166 to reliably block the piping 111, and enables the flow channels to be reliably set.

In the cassette 110 related to the present embodiment, a desired flow channel is formed by attaching the piping 111 to some of the plurality of hooks 113 and strut portions 114. For this reason, it is possible to form flow channels for handling desired radioactive isotopes using one substrate 112. As a result, it is possible to handle a plurality of types of radioactive isotopes using one substrate 112. Additionally, as the hooks 113 and the strut portions 114 are provided corresponding to the elongated hole 116, the piping 111 attached by the hooks 113 and the strut portions 114 can be aligned above and with the elongated holes 116, and a structure capable of opening and closing the piping 111 can be provided.

In the cassette 110 related to the present embodiment, the through holes formed in the substrate 112 are constituted by the elongated holes 116 that extend along the second direction D2, and the plurality of elongated holes 116 are provided at predetermined intervals along the first direction D1. In this case, since the elongated holes 116 as the through holes extend along the second direction D2 and are formed in a wide range, the alignment between the piping 111 and the elongated holes 116 as the through holes becomes easy.

The invention is not limited to the above-described embodiments. The system configuration of the radioactive isotope refining system and the configuration of the solution adjustment unit that are described in the above-mentioned embodiments are merely examples, and may be appropriately changed.

For example, a radioactive isotope refining system 200 as shown in FIG. 17 may be provided. The radioactive isotope refining system 200 is mainly different from the above-described radioactive isotope refining system 100 in that this system includes a refinement unit 240 capable of attaching a cassette 210. In addition, the cassette 210 has the same configuration as the cassette 110 except that the size and shape of a substrate 212 and the configuration of the piping 211 differ. Additionally, the refinement unit 240 have the configuration of the same purport as the solution adjustment unit 102, and includes a fixing portion capable of detachably fixing the cassette 210, and a radioactive isotope refining apparatus 250 having pressing members that press the piping 211 of the cassette 210. In addition, in the refinement unit 240, it is possible to press piping 211 with the pressing members at pressing positions 1R to 1X, 2R to 2X, 3T, 3W, 5T, 5W, 6R to 6X, and 7R to 7X.

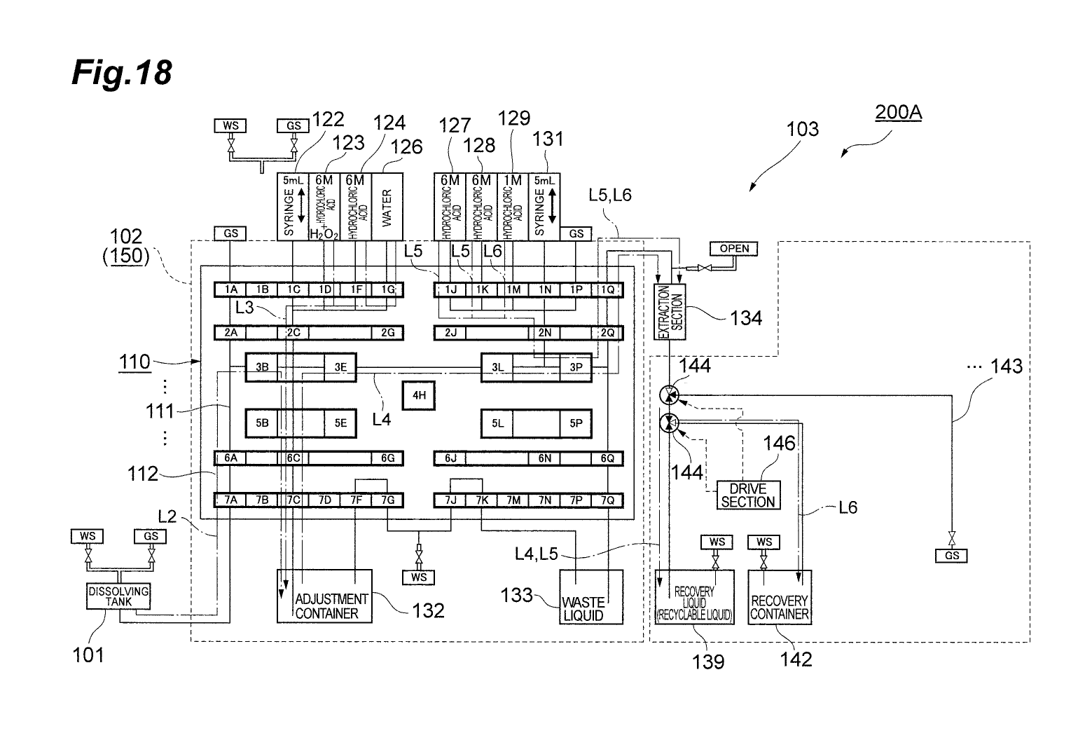

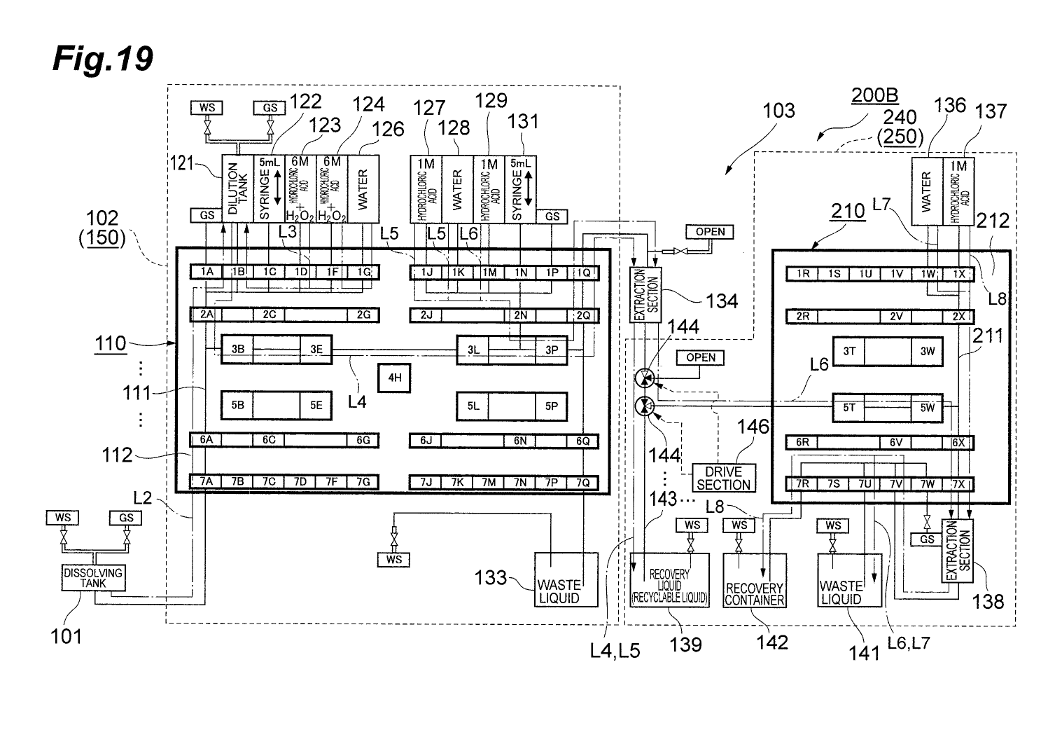

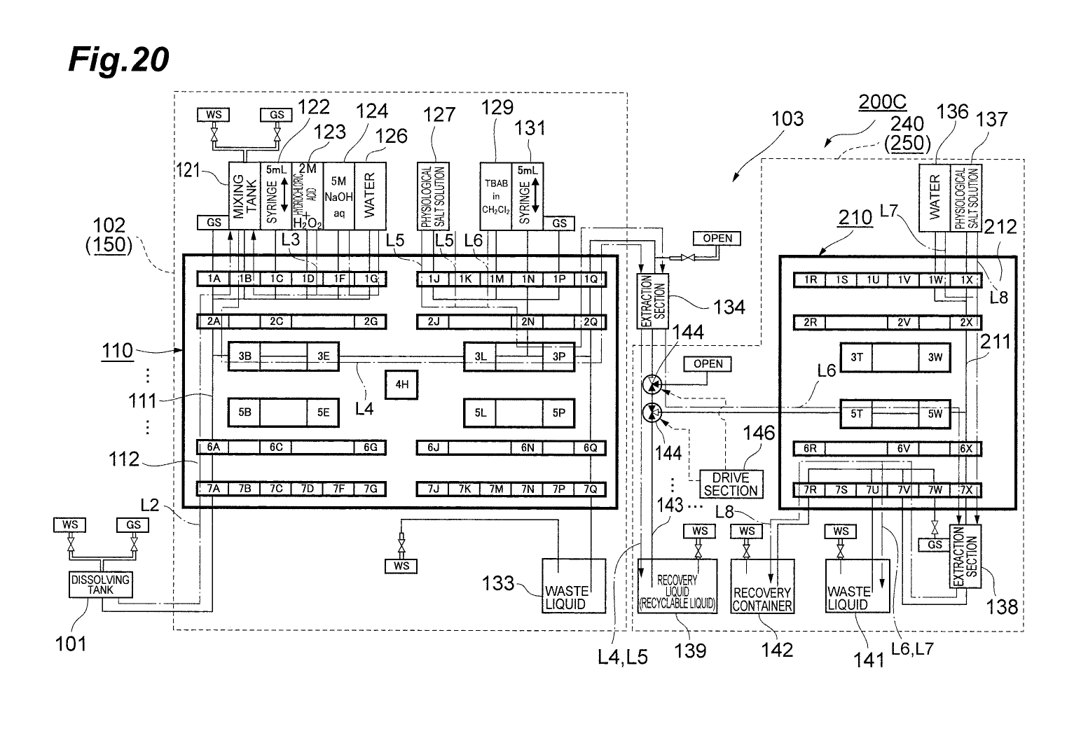

An example of a procedure in a case where radioactive isotopes are refined using the radioactive isotope refining system 200 shown in FIG. 17 will be described. FIG. 18 shows an example of the schematic configuration of a radioactive isotope refining system 200A in a case where ⁶⁴Cu is refined, FIG. 19 shows an example of the schematic configuration of a radioactive isotope refining system 200B in a case where ⁸⁹Zr is refined, and FIG. 20 shows an example of the schematic configuration of a radioactive isotope refining system 200C in a case where ^{99m}Tc is refined.

Refinement of ⁸⁴Cu

First, the radioactive isotope refining system 200A as shown in FIG. 18 is first configured by assembling various piping and containers. The containers to be assembled and the liquids to be contained in these containers are the same as those of the radioactive isotope refining system 100A of FIG. 14. In addition, since the second extraction section 138 and the containers 136 and 137 are not assembled to the refinement unit 240, the cassette 210 is not assembled. Additionally, in the radioactive isotope refining system 200A, the same processing as (Procedure 1 to Procedure 6) of the radioactive isotope refining system 100A of FIG. 14 is performed.

Refinement of ⁶⁹Zr

The radioactive isotope refining system 200B as shown in FIG. 19 is first configured by assembling various piping and containers. The containers to be assembled and the liquids to be contained in these containers are the same as those of the radioactive isotope refining system 100B of FIG. 15. The cassette 210 is attached to the refinement unit 240, and the connectors provided at the respective pieces of piping 211 are connected to the connectors on the side of the containers 136, 137, 141, and 142 and the connectors on the side of the second extraction section 138.

Procedures 1 to 5

In the radioactive isotope refining system 200B, the processing of the same purport as (Procedure 1 to Procedure 5) of the radioactive isotope refining system 100B of FIG. 15 is performed.

Procedures 6 to 8

Although the flow channels of the lines L6 to L8 are set in the radioactive isotope refining system 100B of FIG. 15 by switching the three-way stopcock 144 in the drive section 146, the flow channels of the lines L6 to L8 are set in the radioactive isotope refining system 200B of FIG. 19 by pressing the piping 211 at respective pressing positions. Regarding the others, the processing of the same purport as (Procedure 6 to Procedure 8) of the radioactive isotope refining system 100B of FIG. 15 is performed.

Refinement of ^{99m}Tc

The radioactive isotope refining system 200C as shown in FIG. 20 is first configured by assembling various piping and containers. The containers to be assembled and the liquids to be contained in these containers are the same as those of the radioactive isotope refining system 100C of FIG. 16. The cassette 210 is attached to the refinement unit 240, and the connectors provided at the respective pieces of piping 211 are connected to the connectors on the side of the containers 136, 137, 141, and 142 and the connectors on the side of the second extraction section 138.

Procedures 1 to 5

In the radioactive isotope refining system 200C, the processing of the same purport as (Procedure 1 to Procedure 5) of the radioactive isotope refining system 100C of FIG. 16 is performed.

Procedures 6 to 8

Although the flow channels of the lines L6 to L8 are set in the radioactive isotope refining system 100C of FIG. 16 by switching the three-way stopcock 144 in the drive section 146, the flow channels of the lines L6 to L8 are set in the radioactive isotope refining system 200C of FIG. 20 by pressing the piping 211 at respective pressing positions. Regarding the others, the processing of the same purport as (Procedure 6 to Procedure 8) of the radioactive isotope refining system 100C of FIG. 16 is performed.

The constituent elements that are unitized in the solution adjustment unit and the refinement unit that are described in the above-described embodiments are merely examples, and whether any constituent elements are assembled into a unit may be freely set. For example, the first adjustment container 132 may be assembled into the solution adjustment unit or the refinement unit. Additionally, the solution adjustment unit and the refinement unit may be one unit.

Additionally, the structure of the fixing portion for fixing the cassette are not limited to the embodiments shown in the embodiments, and all structures may be adopted so long as the cassette can be detachably fixed.

It should be understood that the invention is not limited to the above-described embodiments, but may be modified into various forms on the basis of the spirit of the invention. Additionally, the modifications are included in the scope of the invention.

What is claimed is:

1. A cassette for a radioactive isotope handling apparatus comprising:
a substrate including a plurality of holders capable of attaching piping; and
piping attached to the substrate by some of the plurality of holders,
wherein the substrate is provided with a plurality of through holes for opening and closing the piping, and
wherein the plurality of holders include:
a plurality of first holders capable of attaching the piping along a first direction; and
a plurality of second holders capable of attaching the piping along a second direction intersecting the first direction.

2. The cassette for a radioactive isotope handling apparatus according to claim 1,
wherein each of the plurality of first holders is provided on any of a plurality of first lines along the first direction,
wherein each of the plurality of second holders is provided on any of a plurality of second lines along the second direction, and
wherein each of the plurality of through holes is provided so as to be aligned with an intersection point between any of the plurality of first lines and any of the plurality of second lines.

3. The cassette for a radioactive isotope handling apparatus according to claim 1,
wherein at least three of the holders are provided corresponding to each of the through holes.

4. The cassette for a radioactive isotope handling apparatus according to claim 2,
wherein the through holes are constituted by an elongated hole that extends along the second direction, and a plurality of the elongated holes are provided at predetermined intervals along the first direction.

5. A radioactive isotope handling apparatus comprising:
a fixing portion capable of detachably fixing the cassette for a radioactive isotope handling apparatus according to any one of claims 1 to 4; and
a plurality of pressing members that are provided at positions facing the plurality of through holes, respectively, and are capable of pressing the piping.

6. The radioactive isotope handling apparatus according claim 5,
wherein the fixing portion has a front surface that receives the substrate of the cassette for a radioactive isotope handling apparatus, a claw portion that supports the edge of the substrate, and a plurality of protruding portions that protrude from the front surface.

7. The radioactive isotope handling apparatus according to claim 5, further comprising:
a main body portion to which the fixing portion is attached; and
a door portion openably and closably attached to the main body portion,
wherein the plurality of pressing members are provided at the door portion, and are capable of pressing the piping in a state where the door portion is closed.

8. The radioactive isotope handling apparatus according to claim 7,
wherein the main body portion includes protruding portions at positions corresponding to the through holes provided in the cassette for a radioactive isotope handling apparatus, and the pressing members are provided at positions corresponding to the protruding portions.

9. A radioactive isotope handling system comprising:
a fixing portion capable of detachably fixing the cassette for a radioactive isotope handling apparatus according to any one of claims 1 to 4;
a plurality of pressing members that are provided at positions that face the plurality of through holes, respectively, and are capable of pressing the piping;
a solution adjustment unit that performs adjustment of a solution in which radioactive isotopes are dissolved; and
a refinement section that refines the radioactive isotopes contained in the solution adjusted in the solution adjustment unit,
wherein the refinement section has: an extraction section that extracts the radioactive isotopes from the solution;
a replaceable three-way stopcock that is provided downstream of the extraction section; and
a drive section that is provided separately from the three-way stopcock, and applies a driving force for switching of the three-way stopcock.
